(19) United States
(12) Patent Application Publication
(10) Pub. No.: US 2020/0171208 A1
(43) Pub. Date: Jun. 4, 2020

(54) SCAFFOLDS FOR CELL CULTURE AND TISSUE REGENERATION

(71) Applicant: Deakin University, Victoria (AU)

(72) Inventors: Linpeng Fan, Victoria (AU); Jingliang Li, Victoria (AU); Xungai Wang, Victoria (AU)

(21) Appl. No.: 16/624,132

(22) PCT Filed: Jun. 14, 2018
(86) PCT No.: PCT/AU2018/050592

§ 371 Date: Dec. 18, 2019

(30) Foreign Application Priority Data
Jun. 19, 2017 (AU) 2017902326

(51) Int. Cl. A61L 27/48; A61L 27/56; A61L 27/54

(52) U.S. Cl. CPC A61L 27/48; A61L 27/56; A61L 2400/12; A61L 2300/412; A61L 27/54

(57) ABSTRACT

A method for preparing a scaffold, said method comprising the steps of providing a solution comprising fibre-forming molecules; subjecting the solution to a cooling medium to establish a temperature difference at an interface between the cooling medium and solution; and cooling the solution as a result of the temperature difference to induce solvent crystallisation and alignment of fibres in the scaffold.

This application claims priority from Australian Provisional Patent Application No. 2017902326 filed 19 Jun. 2017, the contents of which should be understood to be incorporated.

### TECHNICAL FIELD

The present invention relates to biomaterials and methods for making the same for use in tissue engineering applications such as cell culture, tissue regeneration and wound repair. Scaffolds that mimic natural extracellular matrices for use in tissue engineering and the method of preparing and using said scaffolds having fibres and porosity preferably for cell growth are provided by the invention. Particularly, the methods use a facile strategy for creating hierarchical 3D architectures with co-aligned nanofibres and optionally macrochannels, by manipulating ice crystallization in solutions of macromolecules. The invention also provides for the use of the scaffolds in promoting cell growth and use as a biomedical implant.

### BACKGROUND OF INVENTION

Biomaterials have been of considerable interest in tissue engineering. An ideal biomaterial should provide a biomimetic three-dimensional (3D) environment and support, as well as being able to direct cell behaviour and functions by interaction with cells and mediating the complex multicellular interactions both spatially and temporally. To optimally regulate the cellular fate and activity, biomaterials are continuously being developed to mimic the structural features and functions of natural extracellular matrix (ECM). Natural ECM exists as a 3D porous architecture of intricate nanofibres with diameters ranging between 50 and 500 nm. A main component of the ECM is collagen which has various structural arrangements such as orientation of collagen fibres in different tissues. In a specific tissue, cells are fully responsive to the ECM features to maintain their unique behaviours and functions.

In many tissues with anisotropic structural characteristics (such as dural, tendon, ligament, tympanic and muscle tissues), cells and ECM fibres are highly aligned. These unique alignments support specific physiological functions of tissues and organs. For example, radially aligned nanofibre matrices of the dural and tympanic tissues carry blood and conduct sound, respectively. In skeletal muscle, tendon and ligament tissues, longitudinally aligned fibre bundles support movement and mechanical load. Architectures with aligned nanofibres have been produced in two dimensional (2D) materials using different techniques such as electrospinning and rotary jet spinning. However, these 2D aligned matrices do not mimic the 3D characteristics of native anisotropic tissues and provide support to cells and tissues in a 3D space. Additionally, a drawback of 2D aligned materials are that they have very small pore sizes and low porosity due to mechanical stretching during the fabrication process.

It is extremely difficult to achieve aligned fibre-based 3D scaffolds, in particular, aligned fibre-based 3D scaffolds with interconnected macropores. Furthermore, it has been challenging to obtain the desired alignment of fibres spatially using currently available technologies (such as tubes with fibre alignment towards the short axis or spheres with fibre alignment towards the centre). Currently, the main forms of aligned fibre-based structures are two-dimensional membranes and tubes with very thin walls (two dimensional) which consist of aligned nanofibres along the long axis of tubes. Additionally, pre-existing 3D scaffolds having random fibre orientation do not have sufficient interconnectivity and pore size.

An ideal material for regenerating anisotropic tissue should have a 3D biomimetic architecture with aligned nanofibres and interconnected macropores to direct cell growth, facilitate transport/exchange of nutrients/oxygen/waste and intercellular communications. Although there has been growing interest in mimicking the natural structural features and functions of ECM, the preparation of scaffolds having high alignment with nanofibres and large pores has been challenging.

Currently, the standard treatment of wounds or damaged tissues has been to use autograft. However, it is often limited by a high risk of infection and insufficient donor sites. Further, an autograft can lead to secondary wounds in donor sites and can cause severe scars in both the application and donor sites.

Accordingly, it is desirable to develop a scaffold which has aligned fibres and sufficient interconnectivity and pore size as a material suitable for use in tissue engineering and a method of preparing and using said scaffolds to promote cell growth and tissue formation in the bulk 3D scaffolds.

The discussion of documents, acts, materials, devices, articles and the like is included in this specification solely for the purpose of providing a context for the present invention. It is not suggested or represented that any or all of these matters formed part of the prior art base or were common general knowledge in the field relevant to the present invention as it existed before the priority date of each claim of this application.

Where the terms “comprise”, “comprises”, “comprised” or “comprising” are used in this specification (including the claims) they are to be interpreted as specifying the presence of the stated features, integers, steps or components, but not precluding the presence of one or more other features, integers, steps or components, or group thereof.

### SUMMARY OF INVENTION

Continuous evolution of scaffolds for tissue engineering has been driven by the desire to recapitulate the structural features and functions of natural extracellular matrix (ECM). However, creating 3D architectures having aligned nanofibres and interconnected macropores to mimic the ECM of anisotropic tissues remains a challenge.

Accordingly, in one aspect of the present invention there is provided a method for preparing a scaffold, said method comprising the steps of: providing a solution comprising fibre-forming molecules; subjecting the solution to a cooling medium to establish a temperature difference at an interface between the cooling medium and solution; and cooling the solution as a result of the temperature difference to induce solvent crystallisation and alignment of fibres in the solution to create the scaffold.

Advantageously, the scaffolds of the present invention having aligned fibres can in certain embodiments promote at least one of adhesion, proliferation and differentiation of cells as the scaffold mimics the structure of natural extracellular matrix.

Accordingly, in yet a further embodiment the present invention further comprises subjecting the scaffold to a solution followed by an additional cooling step to induce solvent crystallisation and channels in the scaffold. In certain embodiments, the channels are substantially co-aligned with the aligned fibres.

The channels formed in the scaffold can in certain embodiments promote at least one of cell adhesion (capturing) and proliferation. Channels formed in the scaffold of the present invention can promote three-dimensional cell growth or cell culture for tissue regeneration.

Accordingly, in another aspect, the present invention provides a porous biomimetic scaffold comprising a matrix of substantially aligned fibres. In again another aspect, the present invention provides a porous biomimetic scaffold comprising a three-dimensional matrix of substantially aligned fibres. In yet another aspect, the present invention provides a porous biomimetic scaffold comprising a matrix of fibres. In some embodiments, the fibres are aligned. In some embodiments, the fibres are radially aligned fibres, linearly aligned fibres or longitudinally aligned fibres. In some embodiments, the fibres are unidirectionally aligned.

The scaffolds of the present invention can be used for cell culture and tissue engineering applications. In certain embodiments, the scaffolds provided by the present invention include a method of treating a mammal suffering from a tissue injury and in need of tissue restoration and/or regeneration, comprising applying to the injury site a scaffold of the present invention.

In some embodiments, the inventors have found that the scaffolds are stable in biological systems in certain circumstances and can therefore be used for cell culture, drug delivery, would healing or the treatment of damaged tissue.

### BRIEF DESCRIPTION OF DRAWINGS

FIG. 1 (a) Scaffolds with radially aligned nanofibres and macrochannels. The channel walls are made up of aligned nanofibers along the long axis of the channels as well as pores and particles. (b) Scaffolds with vertically aligned nanofibers and macrochannels.

FIG. 2 Fabricating 3D silk fibroin (SF) scaffolds (A(F&C) scaffolds) with radially co-aligned nanofibres and macrochannels through a facile freeze-drying technology. The hole (in 4. A(F&C) scaffolds above) is a top view of the central channel in the A(F&C) scaffolds. AFb: aligned nanofibrous scaffolds, AF: water-resistant aligned nanofibrous scaffolds without macrochannels and A(F&C): water-resistant scaffolds with radically co-aligned nanofibres and macrochannels.

FIG. 3 Hierarchical structure of a 3D scaffold with radially aligned nanofibres and channels (A(F&C)). (a) Micro-CT images demonstrating the radially aligned channel structure of the scaffold. Scale bars: 1000 μm. (b) SEM images of channel walls at various magnifications revealing the aligned nanofibrous structure with nanoparticles and pores. (Large arrows indicate the orientation of aligned nanofibres. Particles, pores and aligned nanofibres on the channel wall are indicated by small arrows, respectively. Scale bars: from left to right 10, 2, and 1 μm, respectively. (c) A schematic dimension presentation of the relevant structures.

FIG. 4 Polypropylene porous microfibrous materials modified with locally aligned silk fibroin (SF) nanofibres of the present invention (the nanofibers in a, b and c used 0.0125%, 0.025% and 0.05% (w/v) of silk fibroin solutions, respectively). a′, b′ and c′ are the magnification of a, b, and c, respectively. Scale bars: 200 μm in a, b and c; 10 μm in a′ and b′; 30 μm in c′.

FIG. 5 Polypropylene porous microfibrous materials modified with locally aligned alginate nanofibres using 0.025% (w/v) of an alginate solution (a,b,c; a, b and c are at different magnifications) and locally aligned gelatin nanofibres using 0.025% (w/v) of a gelatin solution (d,e,f; d, e and f are at different magnifications) of the present invention. Scale bars: 100, 10, 1, 200, 20, and 1 μm in a, b, c, d, e and f, respectively.

FIG. 6 3D A(F&C) scaffolds enhance capturing and proliferation of adherent Human Umbilical Vein Endothelial Cells (HUVECs), and direct cell migration and growth by aligned nanofibres and channels. (a) Viability (MTS absorbance index) of HUVECs captured by 3D AF, W, W&F and A(F&C) scaffolds. (b) Viability (MTS absorbance index) of HUVECs in 3D AF, W, W&F and A(F&C) scaffolds after different periods of culture. (c) Scheme illustrating how to read images presented in d. (d) Growth of HUVECs in 3D AF, W, W&F and A(F&C) scaffolds after three days of culture. Scale bars: 25 μm in W, W&F, AF and Inset 1; 75 μm in A(F&C).

FIG. 7 Aligned nanofibres and channels in 3D A(F&C) scaffolds facilitate the formation of CD31-positive vessel-like structures by directing the growth, migration and interaction of adherent HUVECs after 21 days of culture (FIG. 6c illustrates how to read images presented in FIG. 7). (a) Growth and interaction of HUVECs in 3D A(F&C), AF, W&F and W scaffolds. Scale bars: 50 μm in A(F&C), W&F and W; 25 μm in AF. (b) Sequential confocal slices of the channel in A(F&C) shown in (a). Scale bars: 50 μm.

FIG. 8 Aligned nanofibres and channels of 3D A(F&C) scaffolds facilitate capture of the non-adherent Embryonic Dorsal Root Ganglion Neuron cells (DRG), and direct the 3D growth of DRG neurites. (a) Viability (MTS absorbance index) of DRGs captured by 3D AF, W, W&F and A(F&C) scaffolds. (b) Confocal fluorescence microscopic images that reveal the structure of W, W&F and AF scaffolds limits DRGs and DRG neurites to grow on the surface of scaffolds. Scale bars: 100 μm for W and W&F scaffolds; 50 μm for AF scaffolds. (c) Aligned nanofibres and channels direct the 3D growth of DRG neurites in 3D A(F&C) scaffolds. Scale bars: from left to right 75, 25 and 25 μm, respectively.

FIG. 9 3D A(F&C) scaffolds direct the growth, migration and interaction of both adherent HUVECs, and non-adherent DRGs and DRG neurites by radially aligned channels and nanofibres. Adherent HUVECs are mainly guided by aligned nanofibres, and non-adherent DRGs and DRG neurites are mainly directed by aligned channels. (a) HUVECs growing and interacting along the aligned nanofibres on channel walls. (b) HUVECs assemble into CD31-positive vessel-like structures along the aligned nanofibres on channel walls. (c), (d) and (e) DRGs and DRG neurites growing along the aligned channels, suggesting the 3D growth of DRGs and DRG neurites in A(F&C) scaffolds. All scale bars are 25 μm.

FIG. 10 (a) Representative SEM images showing aligned nanofibres and nanoparticles in AFb scaffolds. Fast Fourier Transform (FFT) pattern in the inset suggests these nanofibres were well aligned in the radial direction. Scale bars: from left to right 2, 1 and 10 μm, respectively. (b) Directional freezing of aqueous silk fibroin solution in liquid nitrogen allows fabricating 3D silk fibroin nanofibrous scaffolds with various geometries (including cylinders, tubes and particles or spheres), diameters and thicknesses as well as different nanofibre alignments.

FIG. 11 Effects of freezing temperature on the morphology structure of 3D silk fibroin scaffolds. (a) SEM images reveal freezing aqueous silk fibroin at −80° C. leading to 3D scaffolds (W&Fb) with a hybrid structure with short channels/pores/fibres. Scale bars: from left to right 200, 30 and 100 μm, respectively. (b) SEM images show freezing aqueous silk fibroin at −20° C. producing 3D scaffolds (Wb) with wall-like porous structure. Scale bars: from left to right 200, 20 and 100 μm, respectively.

FIG. 12 Representative images of A(F&C) scaffolds from SF/gelatin mixture (a); sodium alginate (b). Red arrows indicate the channels in scaffolds with aligned nanofibres on the wall of channels. Scale bars: 20 μm in a and 2 μm in inset 1, b and inset 2.

FIG. 13 Micro-CT images of the hybrid structure (containing short channels/pores/nanofibres) of W&F and the wall-like porous structure of W 3D scaffolds. The details in structure can be seen clearly in FIG. 14. All scale bars are 1000 μm.

FIG. 14 (a) SEM images of the water-resistant W&F scaffolds after post-treatment. Scale bars: from left to right 100, 20 and 100 μm, respectively. (b) SEM images of the water-resistant W scaffolds after post-treatment. Scale bars: from left to right 100, 20 and 100 μm, respectively.

FIG. 15 ATR-FTIR spectra of 3D silk fibroin scaffolds. (a) ATR-FTIR spectra of silk fibroin scaffolds from different freezing-temperatures: −20° C. (Wb), −80° C. (W&Fb) and liquid nitrogen (AFb). (b) ATR-FTIR spectra of post-treated silk fibroin scaffolds. All scaffolds (A(F&C), W&F and W) present peaks at around 1517, 1622 and 1700 cm⁻¹, suggesting the post-treatment made the structure transition of silk fibroin from random coils to β-sheets.

FIG. 16 (a) Compressive modulus of 3D W, W&F and A(F&C) silk fibroin scaffolds. (b) Morphology of scaffolds after mechanical test. Of note, after being compressed in the mechanical test, A(F&C) scaffolds still maintained a good radially aligned morphology and structure, and just some minor collapses are seen on the surface of scaffolds, probably resulting from damage of some channels.

FIG. 17 Growth of DRGs in W and W&F scaffolds after 21 days of culture. The extension and outgrowth of DRG neurites in W and W&F scaffolds are blocked by surrounding materials, suggesting the scaffolds do not provide DRGs with a suitable 3D environment. Scale bars: 100 and 25 μm in W and W&F, respectively.

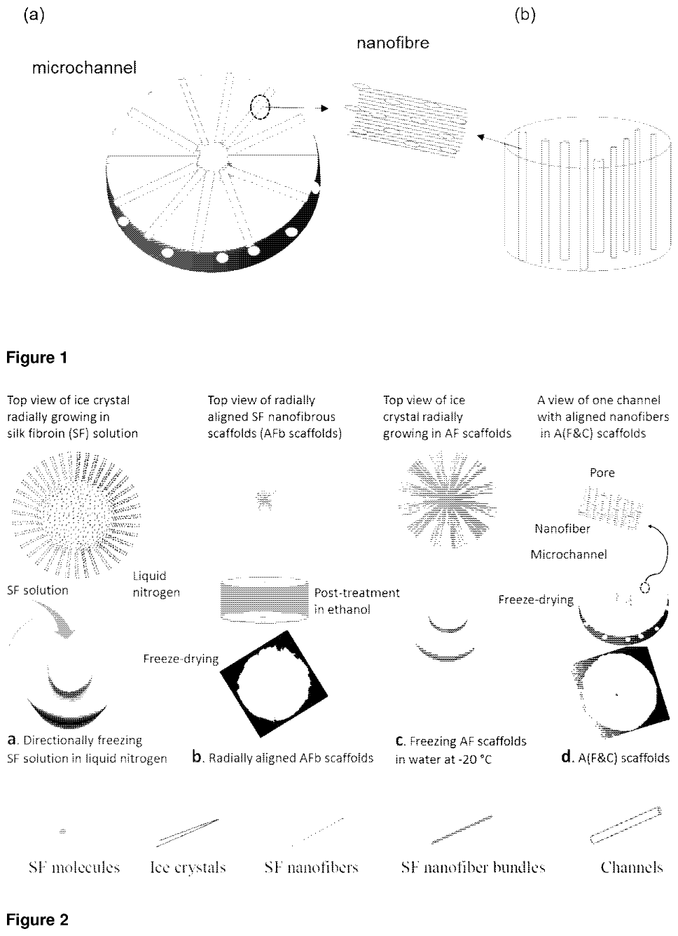

### DETAILED DESCRIPTION

Continuous evolution of scaffolds for tissue engineering has been driven by the desire to recapitulate the structural features and functions of natural extracellular matrix (ECM). However, creating scaffolds having aligned nanofibres and interconnecting macropores to mimic the ECM of anisotropic tissues remains a challenge, particularly in developing 3D scaffolds.

Scaffolds with Aligned Fibres

Accordingly, in one aspect of the present invention there is provided a method for preparing a scaffold, said method comprising the steps of: providing a solution comprising fibre-forming molecules; subjecting the solution to a cooling medium to establish a temperature difference at an interface between the cooling medium and solution; and cooling the solution as a result of the temperature difference to induce solvent crystallisation and alignment of fibres in the solution to create the scaffold.

The present inventors have found that controlled cooling of a solution comprising fibre-forming molecules induces solvent crystallization in which fibres can align to create a scaffold. The alignment of the fibres can be directionally controlled so that crafted scaffolds may be generated having fibres aligned in a direction in which the solvent crystallization forms.

The method of the invention can be used to prepare any “scaffold” which as used herein preferably refers to a three-dimensional matrix of fibres which is suitable as a template for a cell carrier for cell culture, tissue repair, tissue engineering or related applications. Preferably the scaffold is a 3D scaffold comprising channels and pores that enable and facilitate cell culture and flow of biochemical and physicochemical factors within the scaffold which are necessary for cell culture and survival.

The scaffolds are formed from a solution comprising fibre forming molecules. The technique used to prepare a scaffold according to the method of the present invention will depend on the solution, fibre-forming molecule and cooling medium used. It will also be appreciated that the technique used will affect the direction of the alignment of the fibres whether they are longitudinally or radially aligned. The solution may be subjected directly or indirectly to a cooling medium to establish a temperature difference at an interface between the solution and cooling medium. In certain embodiments, the solution comprising fibre-forming molecules is contained in a receptacle prior and subjected indirectly to the solution for cooling.

Alternatively, in some embodiments, the receptacle may be immersed in the cooling medium followed by addition of the solution comprising fibre-forming molecules to the receptacle to induce alignment of fibres. Any suitable receptacle material can be used in the present invention providing a temperature difference is set up at an interface between the solution and the cooling medium. In some embodiments, the receptacle material is selected from but not limited to glass, metal, plastic, ceramic or combinations thereof.

In certain embodiments, the solution comprising fibre-forming molecules can be subjected to a cooling medium directly. For example, the solution comprising fibre-forming molecules can be dripped, sprayed or injected directly into a cooling medium to establish a temperature difference at an interface between the cooling medium and solution to induce solvent crystallization and alignment of fibres in the scaffold.

Without wishing to be bound by any one theory, the inventors believe that the alignment of fibres is controlled by solvent crystallization which occurs when a temperature difference between the solution and the cooling medium is sufficient for nucleation of crystals to form. For instance, where the solvent is water, ice nucleation will form when the temperature difference is sufficient to cause freezing and ice crystals so formed radiate from an interface between the solution and the cooling medium into the solution. The solvent crystals and the direction in which they form are believed to act as templates to control the alignment direction of fibres.

The temperature difference is imperative for the formation of solvent crystallization and alignment of fibres. The temperature difference is determined by the difference in temperature between the solution and the cooling medium.

In certain embodiments, the temperature difference is sufficient to promote nucleation of solvent crystals at the interface. The temperature difference can be measured relative to the solution. For example, if the solution had a temperature of 20° C. and the cooling medium had a temperature of −40° C., the temperature difference would be −60° C. relative to the solution. In certain embodiments, the temperature difference is at least −120° C. relative to the solution. In certain embodiments, the temperature difference is at least −196° C. relative to the solution. In certain embodiments, the temperature difference is in a range of from −20° C. to −296° C. relative to the solution. In certain embodiments, the temperature difference is in a range of from −80° C. to −296° C. relative to the solution or −180° C. to −296° C. relative to the solution. In certain embodiments, the temperature difference is in a range of from −120° C. to −296° C. relative to the solution. In certain embodiments, the temperature difference is in a range of from −20° C. to −196° C. relative to the solution or −30° C., −40° C., −50° C. −60° C. or −70° C. relative to the solution. In certain embodiments, the temperature difference is in a range of from −80° C. to −196° C. relative to the solution or −90° C. or −100° C. relative to the solution. In certain embodiments, the temperature difference is in a range of from −100° C. to −196° C. relative to the solution or −110° C. relative to the solution. In certain embodiments, the temperature difference is in a range of from −120° C. to −196° C. relative to the solution or −130° C., −140° C., −150° C. relative to the solution. In certain embodiments, the temperature difference is in a range of from −150° C. to −196° C. relative to the solution or −160° C. relative to the solution. In certain embodiments, the temperature difference is in a range of from −170° C. to −196° C. relative to the solution or −180° C. or −190° C. relative to the solution.

The direction of the alignment of fibres can be controlled by adjusting the direction of the temperature difference (i.e., cooling direction). In some embodiments, the establishment of the temperature difference between the cooling medium and solution comprising fibre-forming molecules induces aligned fibres from the interface between the solution and cooling medium. In some embodiments, the establishment of the temperature difference between the cooling medium and solution comprising fibre-forming molecules induces unidirectionally aligned fibres from the interface between the solution and cooling medium. As used herein the term “unidirectionally aligned fibres” refers to the fibres in the scaffold being oriented towards a single direction. Non-limiting examples of unidirectionally aligned fibres include either fibres which are roughly parallel to each other (linearly aligned) or run roughly towards a point in space (radially aligned). It is to be understood that not every fibre must be oriented towards a single direction, and some deviation in direction is contemplated.

In certain embodiments, the temperature difference is established circumferentially to the solution to induce radially aligned fibres in the scaffold. In certain embodiments, the temperature difference is established along a plane of the interface to induce linearly or longitudinally aligned fibres in the scaffold. Therefore the plane may be parallel or perpendicular to the interface.

As will be appreciated by a person skilled in the relevant art, the temperature difference is a relative measure of the temperature range between the cooling medium and solution comprising fibre-forming molecules. It can also be convenient to express the temperature sufficient to induce alignment of fibres in absolute terms. For example, the temperature of the cooling medium to induce nucleation of solvent crystals for alignment of fibres can be expressed.

In some embodiments, the cooling medium is at a temperature less than −196° C. In some embodiments, the cooling medium is at a temperature of from −80° C. to −196° C. In some embodiments, the cooling medium is at a temperature less than −80° C. or −90° C., −100° C. In some embodiments, the cooling medium is at a temperature of from −100° C. to −196° C. or −110° C. to −196° C. In some embodiments, the cooling medium is at a temperature of from −120° C. to −196° C. or −130° C. to −196° C. In some embodiments, the cooling medium is at a temperature of from −140° C. to −196° C. or −150° C. to −196° C. In some embodiments, the cooling medium is at a temperature of from −160° C. to −196° C. or −170° C. to −196° C., or −180° C. to −196° C.

It will also be appreciated by a person skilled in the relevant art that the rate of cooling of the solution comprising fibre-forming molecules can influence alignment of fibres. In some embodiments, the solution is cooled at a rate of 0.2° C.·s⁻¹ to 260° C.·s⁻¹. In some embodiments, the solution is cooled at a rate of 5° C.·s⁻¹ to 260° C.·s⁻¹ or 10° C.·s⁻¹ to 260° C.·s⁻¹ or 15° C.·s⁻¹ to 260° C.·s⁻¹. In some embodiments, the solution is cooled at a rate of 20° C.·s⁻¹ to 260° C.·s⁻¹ or 25° C.·s⁻¹ to 260° C.·s⁻¹, 30° C.·s⁻¹ to 260° C.·s⁻¹, 35° C.·s⁻¹ to 260° C.·s⁻¹ or 40° C.·s⁻¹ to 260° C.·s⁻¹. In some embodiments, the solution is cooled at a rate of 50° C.·s⁻¹ to 260° C.·s⁻¹ or 60° C.·s⁻¹ to 260° C.·s⁻¹ or 70° C.·s⁻¹ to 260° C.·s⁻¹. In some embodiments, the solution is cooled at a rate of 80° C.·s⁻¹ to 260° C.·s⁻¹ or 90° C.·s⁻¹ to 260° C.·s⁻¹, 100° C.·s⁻¹ to 260° C.·s⁻¹ or 110° C.·s⁻¹ to 260° C.·s⁻¹. In some embodiments, the solution is cooled at a rate of 120° C.·s⁻¹ to 260° C.·s⁻¹ or 130° C.·s⁻¹ to 260° C.·s⁻¹ or 140° C.·s⁻¹ to 260° C.·s⁻¹. In some embodiments, the solution is cooled at a rate of 150° C.·s⁻¹ to 260° C.·s⁻¹ or 160° C.·s⁻¹ to 260° C.·s⁻¹, 170° C.·s⁻¹ to 260° C.·s⁻¹, 180° C.·s⁻¹ to 260° C.·s⁻¹, 190° C.·s⁻¹ to 260° C.·s⁻¹, 200° C.·s⁻¹ to 260° C.·s⁻¹, 210° C.·s⁻¹ to 260° C.·s⁻¹, 220° C.·s⁻to 260° C.·s⁻¹, 230° C.·s⁻¹ to 260° C.·s⁻¹, 240° C.·s⁻¹ to 260° C.·s⁻¹ or 250° C.·s⁻¹ to 260° C.·s⁻¹.

In certain embodiments, the sample of solution comprising fibre-forming molecules can be gradually immersed into the cooling medium to induce alignment of fibres in the scaffold. In certain embodiments, the solution is subjected by immersion in the cooling medium at a rate of 1 to 15 mm·min⁻¹. In certain embodiments, the solution is subjected by immersion in the cooling medium at a rate of 3 to 15 mm·min⁻¹. In certain embodiments, the solution is subjected by immersion in the cooling medium at a rate of 1 to 10 mm·min⁻¹. In certain embodiments, the solution is subjected by immersion in the cooling medium at a rate of 5 to 10 mm·min⁻¹. In certain embodiments, the solution is subjected by immersion in the cooling medium at a rate of 5 to 8 mm·min⁻¹.

Any suitable cooling medium can be used in the method of the present invention to induce alignment of fibres in the scaffold. In theory the cooling medium could be a solid, a liquid or a gas depending on the exact nature of the cooling medium. For example, the cooling medium could be liquid nitrogen, dry ice, air, liquid ethane, liquid CO₂ and combinations thereof. In certain embodiments, the cooling medium is a freezer. In certain embodiments, the cooling medium is dry ice in combination with at least one of tetrachloroethylene, carbon tetrachloride, 1,3-dichlorobenzene, o-xylene, m-toluidine, acetonitrile, pyridine, m-xylene, n-octane, isopropyl ether, acetone, butyl acetate, propyl amine. In some embodiments, the cooling medium is liquid nitrogen in combination with at least one of ethyl acetate, n-butanol, hexane, acetone, toluene, methanol, ethyl ether, cyclohexane, ethanol, ethyl ether, n-pentane, isopentane. Most preferably, the cooling medium is liquid nitrogen.

Deviation in direction of the alignment of the fibres is contemplated. It can be convenient to express the deviation of the alignment of the fibres relative to the surface normal of the interface between the cooling medium and solution comprising fibre-forming molecules. In one embodiment, the fibres are aligned between 0° to 30° to a surface normal of the interface. In one embodiment, the fibres are aligned between 0° to 25° to a surface normal of the interface. In one embodiment, the fibres are aligned between 0° to 20° to a surface normal of the interface. In one embodiment, the fibres are aligned between 0° to 15° to a surface normal of the interface. In one embodiment, the fibres are aligned between 0° to 10° to a surface normal of the interface. In one embodiment, the fibres are aligned between 0° to 5° to a surface normal of the interface.

The formation of solvent crystals can function as a template which provides control of fibre alignment in the scaffold. The diameter of the solvent crystals will depend on the solvent used, cooling rate, and cooling medium used. Any suitable diameter of solvent crystal can be used in the method of the present invention to induce alignment of fibres. In one embodiment, the solvent crystals formed from solvent crystallisation has a diameter from 20 nm to 5 mm, 20 nm to 4 mm, 20 nm to 3 mm, 20 nm to 2 mm or 20 nm to 1 mm. In one embodiment, the solvent crystals formed from solvent crystallisation has a diameter from 1 nm to 500 μm, 10 nm to 400 μm or 10 nm to 300 μm. In one embodiment, the solvent crystals formed from solvent crystallisation has a diameter from 10 nm to 200 μm. In one embodiment, the solvent crystals formed from solvent crystallisation has a diameter from 10 nm to 100 μm. In one embodiment, the solvent crystals formed from solvent crystallisation has a diameter from 10 nm to up to 90 μm, 80 μm, 70 μm, 60 μm, 50 μm, 40 μm, 30 μm, 20 μm or 10 μm. In one embodiment, the solvent crystals formed from solvent crystallisation has a diameter from 10 nm to 5 μm. In one embodiment, the solvent crystals formed from solvent crystallisation has a diameter from 100 μm to 2 mm. In one embodiment, the solvent crystals formed from solvent crystallisation has a diameter from 10 to 3000 nm. In one embodiment, the solvent crystals formed from solvent crystallisation has a diameter from 10 to 3000 nm. In one embodiment, the solvent crystals formed from solvent crystallisation has a diameter from 20 to 2500 nm. In one embodiment, the solvent crystals formed from solvent crystallisation has a diameter from 20 to 2000 nm. In one embodiment, the solvent crystals formed from solvent crystallisation has a diameter from 50 to 2000 nm. In one embodiment, the solvent crystals formed from solvent crystallisation has a diameter from 50 to 1500 nm. In one embodiment, the solvent crystals formed from solvent crystallisation has a diameter from 50 to 1000 nm. In one embodiment, the solvent crystals formed from solvent crystallisation has a diameter from 50 to 700 nm.

The duration of the cooling step can affect the diameter of the solvent crystals and the resulting fibre diameters. Any suitable duration can be used provided that it is sufficient to induce alignment of fibres in the scaffold. In some embodiments, the solution comprising fibre-forming molecules is cooled for less than 10 minutes. In some embodiments, the solution comprising fibre-forming molecules is cooled for less than 20 minutes. In some embodiments, the solution comprising fibre-forming molecules is cooled for less than 30 minutes. In some embodiments, the solution comprising fibre-forming molecules is cooled for less than 1 hour. In some embodiments, the solution comprising fibre-forming molecules is cooled for less than 5 minutes. In some embodiments, the solution comprising fibre-forming molecules is cooled for less than 1 minute.

As will be appreciated by a person skilled in the relevant art, the scaffolds prepared by the method of the present invention can retain the solvent crystals formed from solvent crystallisation. The solvent crystals can be removed from the scaffold using any suitable technique. For example, the scaffold prepared by the method of the present invention can be lyophilized (freeze-dried) to remove the solvent crystals. Alternatively, the solvent crystals can be thawed into solution state after cooling and solvent removed under reduced pressure such as in a vacuum or vacuum drying oven. In some embodiments, the solvent crystals can be removed from the scaffold using a desiccator.

Depending on the fibre-forming molecule used, the scaffold can be water soluble. In some embodiments, the scaffold can be treated to impart water-resistance. The scaffold can be treated using any suitable agent to impart water-resistance. For example, the scaffold can be subjected to the group consisting of ethanol, methanol, genipin, glutaraldehyde, 1-ethyl-3-(3-dimethylaminopropyl) carbodiimide hydrochloride, calcium chloride, water or combination thereof. A skilled addressee would appreciate that ethanol, methanol, genipin, glutaraldehyde, 1-ethyl-3-(3-dimethylaminopropyl) carbodiimide hydrochloride, calcium chloride or water can be in liquid or vapour phase (for example ethanol solution or ethanol vapour). In certain embodiments, the scaffold is water-resistant.

In other embodiments, the scaffold can be treated to induce cross-linking between the aligned fibres. For example, the scaffold can be subjected to glutaraldehyde or electromagnetic radiation to induce cross-linking in the scaffold. In some embodiments, the scaffold can be subjected to at least one of methanol, ethanol, genipin, 1-ethyl-3-(3-dimethylaminopropyl) carbodiimide hydrochloride, calcium chloride, water, plasma radiation or combinations thereof to induce cross-linking in the scaffold. A skilled addressee would appreciate that methanol, ethanol, genipin, 1-ethyl-3-(3-dimethylaminopropyl) carbodiimide hydrochloride, calcium chloride or water can be in liquid or vapour phase (for example ethanol solution or ethanol vapour).

It will be apparent to a person skilled in the relevant art that any suitable solvent can be used to dissolve the fibre-forming molecules to form a solution. In one embodiment, the solvent is water, organic solvent, inorganic nonaqueous solvent and combinations thereof. In one embodiment, the solution comprising fibre-forming molecules is an aqueous solution. When the solution is an aqueous solution, it will be appreciated that the solvent crystals formed from crystallisation are ice crystals.

Suitable organic solvents can be selected from the group consisting of pentane, cyclopentane, hexane, cyclohexane, benzene, toluene, 1,4-dioxane, chloroform, diethyl ether, dichloromethane, tetrahydrofuran, ethyl acetate, acetone, dimethyl formamide, acetonitrile, dimethyl sulfoxide, nitromethane, propylene carbonate, n-butanol, isopropanol, n-propanol, ethanol, methanol, formic acid, acetic acid, hexafluoroisopropanol, trifluoroacetic acid and combinations thereof.

Suitable inorganic solvents can be selected from the group consisting of liquid ammonia, liquid sulfur dioxide, sulfuryl chloride, sulfuryl chloride fluoride, phosphoryl chloride, dinitrogen tetroxide, antimony trichloride, bromine pentafluoride, hydrogen fluoride, neat sulfuric acid, hydrochloric acid, nitric acid, phosphoric acid, boric acid, hydrofluoric acid, hydrobromic acid, perchloric acid, hydroiodic acid and combinations thereof.

In certain embodiments, the solution comprising fibre-forming molecules can include a mixture of two or more miscible solvents such as a mixture of water and an aqueous soluble solvent, a mixture of two or more organic solvents, or a mixture of an organic and an aqueous soluble solvent.

The amount of fibre-forming molecules dissolved in the solution can be any suitable amount and a person skilled in the relevant art would appreciate that the amount dissolved can depend on the solubility of the fibre-forming molecule and the solvent used. In certain embodiments, the solution comprising fibre-forming molecules is in an amount of from 0.001% to 35% w/v. In certain embodiments, the solution comprising fibre-forming molecules is in an amount of from 1% to 20% w/v. In certain embodiments, the solution comprising fibre-forming molecules is in an amount of from 1% to 25% w/v. In certain embodiments, the solution comprising fibre-forming molecules is in an amount of from 1% to 15% w/v. In certain embodiments, the solution comprising fibre-forming molecules is in an amount of from 1% to 10% w/v. In certain embodiments, the solution comprising fibre-forming molecules is in an amount of from 1% to 5% w/v.

The present invention also relates to a porous biomimetic scaffold comprising a three-dimensional matrix of substantially aligned fibres. In some embodiments, the fibres are unidirectionally aligned. In some embodiments, the fibres are radially aligned. In some embodiments, the fibres are linearly or longitudinally aligned.

The diameter of the fibres in the scaffold of the present invention will depend on the solvent, cooling rate, fibre-forming molecule and cooling medium used. In certain embodiments, the diameter of the fibre is from 20 to 5000 nm, 20 to 4000 nm or 20 to 3000 nm. In certain embodiments, the diameter of the fibre is from 20 to up to 2500 nm, 2000 nm or 1500 nm. In certain embodiments, the diameter of the fibre is from 20 to 1000 nm. In certain embodiments, the diameter of the fibre is from 50 to 600 nm. In certain embodiments, the diameter of the fibre is from 20 to 800 nm. In certain embodiments, the diameter of the fibre is from 100 to 500 nm. In certain embodiments, the diameter of the fibre is from 300 to 800 nm. In certain embodiments, the diameter of the fibre is from 300 to 600 nm.

It can also be convenient to describe the fibres in terms of the length of the aligned fibres. In certain embodiments, the aligned fibres have a length of at least 50 nm. In certain embodiments, the aligned fibres have a length of from 50 nm to 50 mm. In certain embodiments, the aligned fibres have a length of from 50 nm to 4 mm. In certain embodiments, the aligned fibres have a length of from 50 nm to 2 mm. In certain embodiments, the aligned fibres have a length of from 50 nm to 500 μm. In certain embodiments, the aligned fibres have a length of from 50 nm to 1000 μm. In certain embodiments, the aligned fibres have a length of from 100 nm to 500 μm. In certain embodiments, the aligned fibres have a length of from 50 nm to 5000 nm. In certain embodiments, the aligned fibres have a length of from 50 nm to 1000 nm. In certain embodiments, the aligned fibres have a length of from 100 nm to 500 nm. In certain embodiments, the aligned fibres have a length of from 50 nm to 500 nm. In certain embodiments, the aligned fibres have a length of from 50 nm to 5 mm. In certain embodiments, the aligned fibres have a length of from 50 nm to 10 mm. In certain embodiments, the aligned fibres have a length of from 50 nm to 20 mm. In certain embodiments, the aligned fibres have a length of from 50 nm to 30 mm. In certain embodiments, the aligned fibres have a length of from 50 nm to 40 mm.

As discussed previously, the scaffold of the present invention is a three-dimensional matrix of fibres suitable for cell culture, tissue repair, tissue engineering or related applications. The scaffolds can have pores of any diameter suitable for cell culture, tissue repair, tissue engineering or related applications. In certain embodiments, the scaffold has pores of diameter from 1 nm to 500 μm or 20 nm to 500 μm. In certain embodiments, the scaffold has pores of diameter from 20 nm to 400 μm. In certain embodiments, the scaffold has pores of diameter from 20 nm to 300 μm. In certain embodiments, the scaffold has pores of diameter from 20 nm to 200 μm. In certain embodiments, the scaffold has pores of diameter from 20 nm to up to 100 μm, 90 μm, 80 μm, 70 μm, 60 μm, 50 μm, 40 μm, 30 μm, 20 μm, 10 μm or 5 μm. In certain embodiments, the scaffold has pores of diameter from 20 to 1500 nm. In certain embodiments, the scaffold has pores of diameter from 50 to 1000 nm. In certain embodiments, the scaffold has pores of diameter from 20 to 800 nm. In certain embodiments, the scaffold has pores of diameter from 50 to 600 nm. In certain embodiments, the scaffold has pores of diameter from 100 to 600 nm. In certain embodiments, the scaffold has pores of diameter from 20 to 600 nm. In certain embodiments, the scaffold has pores of diameter from 20 to 500 nm.

The scaffold of the present invention can also be conveniently described in terms of porosity. The porosity of the scaffold can depend on the fibre-forming molecule and solvent used. The scaffold porosity was calculated as the ratio of the void volume to the total sample volume. Accordingly, in certain embodiments, the scaffold has a porosity of from 0.01% to 95%. In certain embodiments, the scaffold has a porosity of from 20% to 95%, 30% to 95% or 40% to 95%. In certain embodiments, the scaffold has a porosity of from 40% to 90%, 50% to 90%, 60% to 90%, 70% to 90%, 80% to 90% or 85% to 90%. In certain embodiments, the scaffold has a porosity of from 40% to 80%, 40% to 70%, 40% to 60% or 40% to 50%. In certain embodiments, the scaffold has a porosity of from 60% to 80% or 65% to 75%. In certain embodiments, the scaffold has a porosity of from 30% to 60%, 30% to 50% or 30% to 40%.

It will be appreciated that the amount of aligned fibres in the scaffold can vary. This variation of the amount of aligned fibres in the scaffold can be described based on the total dry weight of the scaffold. Accordingly, in some embodiments, at least 5% w/w of the scaffold comprises aligned fibres, based on the total dry weight of the scaffold. In some embodiments, at least 10% w/w, 20% w/w, 30% w/w, 40% w/w, 50% w/w or 60% w/w of the scaffold comprises aligned fibres, based on the total dry weight of the scaffold. In some embodiments, at least 70% w/w of the scaffold comprises aligned fibres, based on the total dry weight of the scaffold. In some embodiments, at least 80% w/w of the scaffold comprises aligned fibres, based on the total dry weight of the scaffold. In some embodiments, at least 90% w/w of the scaffold comprises aligned fibres, based on the total dry weight of the scaffold. In some embodiments, the scaffold comprises of from 50% to 90% w/w of aligned fibres, based on the total dry weight of the scaffold. In some embodiments, the scaffold comprises of from 60% to 90% w/w of aligned fibres, based on the total dry weight of the scaffold. In some embodiments, the scaffold comprises of from 70% to 90% w/w of aligned fibres, based on the total dry weight of the scaffold. In some embodiments, the scaffold comprises of from 80% to 90% w/w of aligned fibres, based on the total dry weight of the scaffold.

As will be appreciated by a person skilled in the relevant art, the scaffold can take any suitable shape and can be for example in the shape of spheres, cubes, prisms, fibres, rods, tetrahedrons, tubes, or irregular particles. As will be appreciated by a person skilled in the relevant art, the shape of the scaffold can be controlled by using a receptacle as discussed above and the shape of the receptacle can typically determine the shape of the scaffold ultimately produced.

Typically, a radially aligned fibre scaffold can be prepared by providing a solution of fibre-forming molecules in a cylindrical sample tube. The sample tube can be immersed in the cooling medium (such as liquid nitrogen) to establish the temperature difference at an interface between the cooling medium and solution circumferentially to induce formation of radially aligned fibres in the scaffold.

Alternatively, a linearly or longitudinally aligned fibre scaffold can be typically prepared by providing a solution of fibre-forming molecules in a cylindrical sample tube having a flat base. The sample tube can be slowly lowered into the cooling medium (such as liquid nitrogen) from the flat base end to establish the temperature difference at an interface between the cooling medium and solution along the plane substantially parallel to the base to induce formation of linearly or longitudinally aligned fibres in the scaffold.

The scaffold can be of any suitable size with the size being determined, in part by the desired size of the scaffold ultimately produced or the size of the receptacle, if used. In certain embodiments, the size of the scaffold can be controlled by mechanical treatment such as cutting the scaffold using a blade or laser. In other embodiments, the scaffold is formed by controlling the cooling of the solution comprising fibre-forming molecules such that as the scaffold is formed, the cooling step is terminated once the desired scaffold size is reached.

The scaffold of the present invention is typically less than 10 cm in at least one dimension. In one embodiment, the scaffold has a size of from 20 nm to 10 cm in at least one dimension. In one embodiment, the scaffold has a size of from 1 mm to 10 cm in at least one dimension. In one embodiment, the scaffold has a size of from 5 mm to 8 cm in at least one dimension. In one embodiment, the scaffold has a size of from 5 mm to 5 cm in at least one dimension. In one embodiment, the scaffold has a size of from 1 mm to 3 cm in at least one dimension. In one embodiment, the scaffold has a size of from 1 mm to 2 cm in at least one dimension. In one embodiment, the scaffold has a size of from 1 mm to 1 cm in at least one dimension.

In certain embodiments, the scaffold of the present invention has a compressive modulus of 5 to 5000 kPa. In certain embodiments, the scaffold of the present invention has a compressive modulus of 5 kPa to up to 4500 kPa, 4000 kPa, 3500 kPa, 3000 kPa, 2500 kPa, 2000 kPa, 1500 kPa, 1000 kPa, 500 kPa, 400 kPa, 300 kPa or 200 kPa. In certain embodiments, the scaffold of the present invention has a compressive modulus of 20 to 160 kPa. In certain embodiments, the scaffold has a compressive modulus of 20 to 140 kPa. In certain embodiments, the scaffold has a compressive modulus of 20 to 120 kPa. In certain embodiments, the scaffold has a compressive modulus of 40 to 100 kPa. In certain embodiments, the scaffold has a compressive modulus of 60 to 100 kPa. In certain embodiments, the scaffold has a compressive modulus of 70 to 100 kPa. In certain embodiments, the scaffold has a compressive modulus of 80 to 100 kPa.

Scaffolds with Aligned Fibres and Channels

In some embodiments, the method of the present invention can further comprise subjecting the scaffold to a solution or solvent followed by an additional cooling step to induce solvent crystallisation and channels in the scaffold. In some embodiments, the channels are substantially co-aligned with the aligned fibres. In some embodiments, the channels can be microchannels or macrochannels.

It is to be understood that the additional cooling step can be at any suitable temperature to induce channels in the scaffold. In one embodiment, the additional cooling step is at a temperature of from −5° C. to −196° C. In one embodiment, the additional cooling step is at a temperature of from −10° C. to −196° C. In one embodiment, the additional cooling step is at a temperature of from −5° C. to −80° C. In one embodiment, the additional cooling step is at a temperature of from −10° C. to −80° C. In one embodiment, the additional cooling step is at a temperature of from −10° C. to −60° C. In one embodiment, the additional cooling step is at a temperature of from −10° C. to −40° C. In one embodiment, the additional cooling step is at a temperature of from −10° C. to −30° C. In one embodiment, the additional cooling step is at a temperature of from −10° C. to −25° C., −11° C. to −25° C., −12° C. to −25° C., −13° C. to −25° C., −14° C. to −25° C., −15° C. to −25° C., −16° C. to −25° C., −17° C. to −25° C., −18° C. to −24° C., −18° C. to −23° C., −18° C. to −22° C. or −19° C. to −21° C.

The present inventors believe that the formation of solvent crystals formed from the additional cooling step induces channel formation in the scaffold. Without wishing to be bound by any one theory, the present inventors believe that use of a higher temperature for the additional cooling step induces larger solvent crystals. In one embodiment, the solvent crystals formed during the additional cooling step have a diameter from 20 nm to 4 mm. In one embodiment, the solvent crystals formed during the additional cooling step have a diameter from 100 μm to 2 mm. In one embodiment, the solvent crystals formed during the additional cooling step have a diameter from 50 nm to 1000 nm. In one embodiment, the solvent crystals formed during the additional cooling step have a diameter from 100 μm to 2 mm. In one embodiment, the solvent crystals formed during the additional cooling step have a diameter from 100 μm to 1000 μm. In one embodiment, the solvent crystals formed during the additional cooling step have a diameter from 500 μm to 1000 μm.

As will be appreciated, the duration of the additional cooling step can affect the diameter of the solvent crystals and the resulting channel diameters. Any suitable duration can be used provided that it is sufficient to induce channel formation in the scaffold. In some embodiments, the additional cooling step is performed between 5 minutes to 96 hours. In some embodiments, the additional cooling step is performed between 10 minutes to 60 hours. In some embodiments, the additional cooling step is performed between 1 hour to 96 hours. In some embodiments, the additional cooling step is performed between 1 hour to 60 hours. In some embodiments, the additional cooling step is performed between 12 hours to 50 hours. In some embodiments, the additional cooling step is performed between 24 hours to 48 hours. In some embodiments, the additional cooling step is performed between 36 hours to 50 hours. In some embodiments, the additional cooling step is performed between 48 hours to 60 hours.

As discussed previously, in certain embodiments, the scaffold further comprises a channel. The diameter of the channels can vary depending on the fibre-forming molecule, solvent, duration of the additional cooling step and solvent crystal diameter. In one embodiment, the channel has a diameter from 20 nm to 2 cm, 20 nm to 1 cm, 20 nm to 500 μm, 20 nm to 400 μm, 20 nm to 300 μm, 20 nm to 200 μm or 20 nm to 100 μm. In one embodiment, the channel has a diameter from 10 μm to 4 mm, 10 μm to 3 mm, 10 μm to 2 mm or 10 μm to 1 mm. In some embodiments, the channel has a diameter of from 20 nm to 4 mm. In some embodiments, the channel has a diameter of from 10 μm to 2 mm. In some embodiments, the channel has a diameter of from 50 μm to 1 mm. In some embodiments, the channel has a diameter of from 100 μm to 1000 μm. In some embodiments, the channel has a diameter of from 100 μm to 800 μm. In some embodiments, the channel has a diameter of from 100 μm to 600 μm. In some embodiments, the channel has a diameter of from 100 μm to 400 μm. In some embodiments, the channel has a diameter of from 20 nm to 2 mm. In some embodiments, the channel has a diameter of from 20 nm to 1 mm. In some embodiments, the channel has a diameter of from 400 μm to 1000 μm. In some embodiments, the channel has a diameter of from 400 μm to 800 μm.

Advantageously, the present inventors have found that in embodiments where the scaffold comprises aligned fibres and channels in the scaffold, the scaffolds of the present invention had significantly higher cell viability than scaffolds comprising aligned fibres without channels. In some embodiments, the scaffold comprising aligned fibres and channels showed improved cell capturing and proliferation. In some embodiments, the aligned fibres and co-aligned channels can direct migration of cells and infiltration of tissues, and thus accelerate the regeneration or function reestablishment of damaged tissues. The scaffolds of the present invention can be useful for repair of wounds (radial growth of tissue can assist wound closure) and can assist in repair of cracked bones.

### Fibre-Forming Molecules

The scaffold of the present invention and the method of preparing the same can be prepared using any suitable fibre-forming molecule. In some embodiments, the fibre-forming molecules are selected from the group consisting of a natural polymer, a synthetic polymer and combinations thereof.

Natural polymers may include polysaccharides, polypeptides, glycoproteins, and derivatives thereof and copolymers thereof. Polysaccharides may include agar, alginates, chitosan, hyaluronan, cellulosic polymers (e.g., cellulose and derivatives thereof as well as cellulose production by-products such as lignin) and starch polymers. Polypeptides may include various proteins, such as silk fibroin, lysozyme, collagen, keratin, casein, gelatin and derivatives thereof. Derivatives of natural polymers, such as polysaccharides and polypeptides, may include various salts, esters, ethers, and graft copolymers. Exemplary salts may be selected from sodium, zinc, iron and calcium salts.

In certain embodiments, the natural polymer is selected from the group consisting of at least one of silk fibroin, alginate, bovine serum albumin, collagen, chitosan, gelatin, sericin, hyaluronic acid, starch and derivatives thereof. In certain embodiments, the natural polymer is selected from the group consisting of silk fibroin, alginates, gelatin, silk fibroin/alginate, silk fibroin/bovine serum albumin, silk fibroin/collagen, silk fibroin/chitosan, silk fibroin/gelatin and derivatives thereof.

Synthetic polymers may include vinyl polymers such as, but not limited to, polyethylene, polypropylene, poly(vinyl chloride), polystyrene, polytetrafluoroethylene, poly(α-methylstyrene), poly(acrylic acid), poly(methacrylic acid), poly(isobutylene), poly(acrylonitrile), poly(methyl acrylate), poly(methyl methacrylate), poly(acrylamide), poly(methacrylamide), poly(1-pentene), poly(1,3-butadiene), poly(vinyl acetate), poly(2-vinyl pyridine), poly(vinyl alcohol), poly(vinyl pyrrolidone), poly(styrene), poly(styrene sulfonate) poly(vinylidene hexafluoropropylene), 1,4-polyisoprene, and 3,4-polychloroprene. Suitable synthetic polymers may also include non-vinyl polymers such as, but not limited to, poly(ethylene oxide), polyformaldehyde, polyacetaldehyde, poly(3-propionate), poly(10-decanoate), poly(ethylene terephthalate), polycaprolactam, poly(11-undecanoamide), poly(hexamethylene sebacamide), poly(m-phenylene terephthalate), poly(tetramethylene-m-benzenesulfonamide). Copolymers of any one of the aforementioned may also be used.

Synthetic polymers employed in the process of the invention may fall within one of the following polymer classes: polyolefins, polyethers (including all epoxy resins, polyacetals, poly(orthoesters), polyetheretherketones, polyetherimides, poly(alkylene oxides) and poly(arylene oxides)), polyamides (including polyureas), polyamideimides, polyacrylates, polybenzimidazoles, polyesters (e.g. polylactic acid (PLA), polyglycolic acid (PGA), poly(lactic-co-glycolic acid) (PLGA)), polycarbonates, polyurethanes, polyimides, polyamines, polyhydrazides, phenolic resins, polysilanes, polysiloxanes, polycarbodiimides, polyimines (e.g. polyethyleneimine), azo polymers, polysulfides, polysulfones, polyether sulfones, oligomeric silsesquioxane polymers, polydimethylsiloxane polymers and copolymers thereof.

In some embodiments, functionalised synthetic polymers may be used. In such embodiments, the synthetic polymers may be modified with one or more functional groups. Examples of functional groups include boronic acid, alkyne or azido functional groups. Such functional groups will generally be covalently bound to the polymer. The functional groups may allow the polymer to undergo further reaction, or to impart additional properties to the fibres.

In some embodiments, the fibre-forming liquid includes a water-soluble or water-dispersible polymer, or a derivative thereof. In some embodiments, the fibre-forming liquid is a polymer solution including a water-soluble or water-dispersible polymer, or a derivative thereof, dissolved in an aqueous solvent. Exemplary water-soluble or water-dispersible polymers that may be present in a fibre-forming liquid such as a polymer solution may be selected from the group consisting of polypeptides, alginates, chitosan, starch, collagen, polyurethanes, polyacrylic acid, polyacrylates, polyacrylamides (including poly(N-alkyl acrylamides) such as poly(N-isopropyl acrylamide), poly(vinyl alcohol), polyallylamine, polyethyleneimine, poly(vinyl pyrrolidone), poly(lactic acid), poly(ethylene-co-acrylic acid), and copolymers thereof and combinations thereof. Derivatives of water-soluble or water-dispersible polymers may include various salts thereof.

In some embodiments, the fibre-forming liquid includes an organic solvent soluble polymer. In some embodiments, the fibre-forming liquid is a polymer solution including an organic solvent soluble polymer dissolved in an organic solvent. Exemplary organic solvent soluble polymers that may be present in a fibre-forming liquid such as a polymer solution include poly(styrene) and polyesters such as poly(lactic acid), poly(glycolic acid), poly(caprolactone) and copolymers thereof, such as poly(lactic-co-glycolic acid).

In some embodiments, the fibre-forming liquid includes hybrid polymer. Hybrid polymers may be inorganic/organic hybrid polymers. Exemplary hybrid polymers include polysiloxanes, such as poly(dimethylsiloxane) (PDMS).

In some embodiments the fibre-forming liquid includes at least one polymer selected from the group consisting of polypeptides, alginates, chitosan, starch, collagen, silk fibroin, polyurethanes, polyacrylic acid, polyacrylates, polyacrylamides, polyesters, polyolefins, boronic acid functionalised polymers, polyvinylalcohol, polyallylamine, polyethyleneimine, poly(vinyl pyrrolidone), poly(lactic acid), polyether sulfone and inorganic polymers.

In some embodiments, the fibre-forming liquid includes a mixture of two or more polymers, such as a mixture of a thermoresponsive synthetic polymer (e.g. poly(N-isopropyl acrylamide)) and a natural polymer (e.g. a polypeptide). The use of polymer blends may be advantageous as it provides avenues for fabricating polymer fibres with a range of physical properties (e.g. thermoresponsive and biocompatible or biodegradable properties). The process of the invention can therefore be used to form aligned fibres with tuneable or tailored physical properties by selection of an appropriate blend or mixture of polymers.

Polymers used in the process of the invention can include homopolymers of any of the foregoing polymers, random copolymers, block copolymers, alternating copolymers, random tripolymers, block tripolymers, alternating tripolymers, derivatives thereof (e.g., salts, graft copolymers, esters, or ethers thereof), and the like. The polymer may be capable of being crosslinked in the presence of a multifunctional crosslinking agent.

Fibre-forming molecules employed in the process may be of any suitable molecular weight and molecular weight is not considered a limiting factor provided the method of the invention can align fibres in the scaffold. The number average molecular weight may range from a few hundred Dalton (e.g. 250 Da) to more several thousand Dalton (e.g. more than 10,000 Da), although any molecular weight could be used without departing from the invention. In some embodiments, the number average molecular weight may be in the range of from about 50 to about 1×10⁷. In some embodiments, the number average molecular weight may be in the range of from about 1×10⁴ to about 1×10⁷.

### Additives

The scaffold of the present invention and the method of preparing the same can comprise an additive. Any suitable additive can be added to impart functionality to the scaffold such as having desired biological activity, improving solubility of the fibre-forming molecule or promoting formation of fibres and/or channels in the scaffolds. In some embodiments, the additive is selected from the group consisting of a drug, growth factor, polymer, surfactant, chemical, particle, porogen and combinations thereof.

The additive can be added in the scaffolds of the present invention in any way known in the art. In one embodiment, the additive can be added in the scaffold by dissolving or dispersing the additive in the solution comprising fibre-forming molecules. The scaffold formed using the method of the present invention would encapsulate the additive during the cooling step. In another embodiment, the additive can be added in the scaffold during the additional cooling step. The additive can be added by subjecting the scaffold to a solution comprising the additive followed by the additional cooling step to induce solvent crystallisation and channels in the scaffold. In another embodiment, the additive in solution is brought into contact with the scaffold such that a certain amount of the additive in solution is adsorbed, absorbed or dispersed into the pores of the scaffold. Adsorption or absorption of the additive in solution can be added in the scaffold by any suitable technique known in the art such as dialysis. In certain embodiments, the additive can be added in the scaffold by chemical reactions (such as catalysis in the scaffold to introduce the desired additive).

As used herein the term “drug” refers a molecule, group of molecules, complex, substance or derivative thereof administered to an organism for diagnostic, therapeutic, preventative medical, or veterinary purposes.

The drug can act to control infection or inflammation, enhance cell growth and tissue regeneration, control tumor growth, act as an analgesic, promote anti-cell attachment, and enhance bone growth, among other functions. Other suitable drugs can include anti-viral agents, hormones, antibodies, or therapeutic proteins. Other drugs include prodrugs, which are agents that are not biologically active when administered but, upon administration to a subject are converted to drugs through metabolism or some other mechanism.

Drugs can also specifically include nucleic acids and compounds comprising nucleic acids that produce a bioactive effect, for example deoxyribonucleic acid (DNA), ribonucleic acid (RNA), or mixtures or combinations thereof, including, for example, DNA nanoplexes. Drugs include the herein disclosed categories and specific examples. It is not intended that the category be limited by the specific examples. Those of ordinary skill in the art will recognize also numerous other compounds that fall within the categories and that are useful according to the invention.

Examples of drugs include a radiosensitizer, a steroid, a xanthine, a beta-2-agonist bronchodilator, an anti-inflammatory agent, an analgesic agent, a calcium antagonist, an angiotensin-converting enzyme inhibitors, a beta-blocker, a centrally active alpha-agonist, an alpha-1-antagonist, an anticholinergic/antispasmodic agent, a vasopressin analogue, an antiarrhythmic agent, an antiparkinsonian agent, an antiangina/antihypertensive agent, an anticoagulant agent, an antiplatelet agent, a sedative, an ansiolytic agent, a peptidic agent, a biopolymeric agent, an antineoplastic agent, a laxative, an antidiarrheal agent, an antimicrobial agent, an antifingal agent, a vaccine, a protein, or a nucleic acid. In other embodiments, the drug can be coumarin, albumin, steroids such as betamethasone, dexamethasone, methylprednisolone, prednisolone, prednisone, triamcinolone, budesonide, hydrocortisone, and pharmaceutically acceptable hydrocortisone derivatives; xanthines such as theophylline and doxophylline; beta-2-agonist bronchodilators such as salbutamol, fenterol, clenbuterol, bambuterol, salmeterol, fenoterol; antiinflammatory agents, including antiasthmatic anti-inflammatory agents, antiarthritis antiinflammatory agents, and non-steroidal antiinflammatory agents, examples of which include but are not limited to sulfides, mesalamine, budesonide, salazopyrin, diclofenac, pharmaceutically acceptable diclofenac salts, nimesulide, naproxene, acetaminophen, ibuprofen, ketoprofen and piroxicam; analgesic agents such as salicylates; calcium channel blockers such as nifedipine, amlodipine, and nicardipine; angiotensin-converting enzyme inhibitors such as captopril, benazepril hydrochloride, fosinopril sodium, trandolapril, ramipril, lisinopril, enalapril, quinapril hydrochloride, and moexipril hydrochloride; beta-blockers (i.e., beta adrenergic blocking agents) such as sotalol hydrochloride, timolol maleate, esmolol hydrochloride, carteolol, propanolol hydrochloride, betaxolol hydrochloride, penbutolol sulfate, metoprolol tartrate, metoprolol succinate, acebutolol hydrochloride, atenolol, pindolol, and bisoprolol fumarate; centrally active alpha-2-agonists such as clonidine; alpha-1-antagonists such as doxazosin and prazosin; anticholinergic/antispasmodic agents such as dicyclomine hydrochloride, scopolamine hydrobromide, glycopyrrolate, clidinium bromide, flavoxate, and oxybutynin; vasopressin analogues such as vasopressin and desmopressin; antiarrhythmic agents such as quinidine, lidocaine, tocainide hydrochloride, mexiletine hydrochloride, digoxin, verapamil hydrochloride, propafenone hydrochloride, flecainide acetate, procainamide hydrochloride, moricizine hydrochloride, and disopyramide phosphate; antiparkinsonian agents, such as dopamine, L-Dopa/Carbidopa, selegiline, dihydroergocryptine, pergolide, lisuride, apomorphine, and bromocryptine; antiangina agents and antihypertensive agents such as isosorbide mononitrate, isosorbide dinitrate, propranolol, atenolol and verapamil; anticoagulant and antiplatelet agents such as coumadin, warfarin, acetylsalicylic acid, and ticlopidine; sedatives such as benzodiazapines and barbiturates; ansiolytic agents such as lorazepam, bromazepam, and diazepam; peptidic and biopolymeric agents such as calcitonin, leuprolide and other LHRH agonists, hirudin, cyclosporin, insulin, somatostatin, protirelin, interferon, desmopressin, somatotropin, thymopentin, pidotimod, erythropoietin, interleukins, melatonin, granulocyte/macrophage-CSF, and heparin; antineoplastic agents such as etoposide, etoposide phosphate, cyclophosphamide, methotrexate, 5-fluorouracil, vincristine, doxorubicin, cisplatin, hydroxyurea, leucovorin calcium, tamoxifen, flutamide, asparaginase, altretamine, mitotane, and procarbazine hydrochloride; laxatives such as senna concentrate, casanthranol, bisacodyl, and sodium picosulphate; antidiarrheal agents such as difenoxine hydrochloride, loperamide hydrochloride, furazolidone, diphenoxylate hdyrochloride, and microorganisms; vaccines such as bacterial and viral vaccines; antimicrobial agents such as penicillins, cephalosporins, and macrolides, antifungal agents such as imidazolic and triazolic derivatives; and nucleic acids such as DNA sequences encoding for biological proteins, and antisense oligonucleotides.

Growth factors as additives suitable in the present invention can stimulate cell growth, proliferation, healing or differentiation. The growth factor can be a protein or steroid hormone. For example, the growth factors can be bone morphogenetic proteins to stimulate bone cell differentiation. Further, fibroblast growth factors and vascular endothelial growth factors can stimulate blood vessel differentiation (angiogenesis).

Growth factors can be selected from the group consisting of adrenomedullin, angiopoietin, autocrine motility factor, bone morphogenetic proteins, ciliary neurotrophic factor family (such as ciliary neurotrophic factor, leukemia inhibitory factor, interleukin-6), colony-stimulating factors (such as macrophage colony-stimulating factor, granulocyte colony-stimulating factor and granulocyte macrophage colony-stimulating factor), epidermal growth factor, ephrins (such as ephrin A1, ephrin A2, ephrin A3, ephrin A4, ephrin A5, ephrin B1, ephrin B2 and ephrin B3), erythropoietin, fibroblast growth factor (such as fibroblast growth factor 1, fibroblast growth factor 2, fibroblast growth factor 3, fibroblast growth factor 4, fibroblast growth factor 5, fibroblast growth factor 6, fibroblast growth factor 7, fibroblast growth factor 8, fibroblast growth factor 9, fibroblast growth factor 10, fibroblast growth factor 11, fibroblast growth factor 12, fibroblast growth factor 13, fibroblast growth factor 14, fibroblast growth factor 15, fibroblast growth factor 16, fibroblast growth factor 17, fibroblast growth factor 18, fibroblast growth factor 19, fibroblast growth factor 20, fibroblast growth factor 21, fibroblast growth factor 22 and fibroblast growth factor 23), foetal bovine somatotrophin, GDNF family of ligands (such as glial cell line-derived neurotrophic factor (GDNF), neurturin, persephin and artemin), growth differentiation factor-9, hepatocyte growth factor, hepatoma-derived growth factor, insulin, insulin-like growth factors (such as insulin-like growth factor-1 and insulin-like growth factor-2), interleukins (such as IL-1, IL-2, IL-3, IL-4, IL-5, IL-6 and IL-7), keratinocyte growth factor, migration-stimulating factor, macrophage-stimulating protein, myostatin, neuregulins (such as neuregulin 1, neuregulin 2, neuregulin 3 and neuregulin 4), neurotrophins (such as brain-derived neurotrophic factor, nerve growth factor, neurotrophin-3, neurotrophin-4), placental growth factor, platelet-derived growth factor, renalase, T-cell growth factor, thrombopoietin, transforming growth factors (such as transforming growth factor alpha and transforming growth factor beta), tumor necrosis factor-alpha, vascular endothelial growth factor and combinations thereof.

The scaffolds can also contain adjuvants such as preservative, wetting agents, emulsifying agents, and dispersing agents. Prevention of the action of micro-organisms may be ensured by the inclusion of various antibacterial and antifungal agents, for example, paraben, chlorobutanol, phenol sorbic acid, and the like. It may also be desirable to include isotonic agents such as sugars, sodium chloride, and the like.

It would be appreciated by a person skilled in the relevant art that polymers suitable as additives in the present invention can be the polymers as already discussed above in relation to the fibre-forming molecule.

Surfactants as additives suitable in the present invention can increase the solubility of the fibre-forming molecules. Without wishing to be bound by any one theory, the present inventors believe that the surfactants can reduce self-aggregation of the fibre-forming molecules to increase the solubility of the solution comprising fibre-forming molecules. In one embodiment, the surfactant is anionic, cationic, zwitterionic or non-ionic. In one embodiment, the surfactant comprises a functional group selected from the group consisting of sulfate, sulfonate, phosphate, carboxylate, amine, ammonium, alcohol, ether and combination thereof. In one embodiment, the surfactant is selected from the group consisting of sodium stearate, sodium dodecyl sulfate, cetrimonium bromide, 4-(5-dodecyl) benzenesulfonate, 3-[(3-cholam idopropyl)dimethylam monio]-1-propanesulfonate, phosphatidylserine, phosphatidylethanolamine, phosphatidylcholine, octaethylene glycol monododecyl ether, pentaethylene glycol monododecyl ether, decyl glucoside, lauryl glucoside, octyl glucoside, triton X-100, nonoxynol-9, glyceryl laurate, polysorbate, dodecyldimethylamine oxide, polysorbate (such as polysorbate 20 and polysorbate 80; sold commercially as Tween 20 and Tween 80), cocamide monoethanolamine, cocamide diethanolamine, poloxamer, polyethoxylated tallow amine and combinations thereof.

Scaffolds with Aligned Fibres and Central Channel

In certain embodiments, the scaffold of the present invention can further comprise a central channel. The central channel can be directed along an axis of the scaffold such as the longitudinal axis of the scaffold. The central channel can be formed using any suitable technique known in the art. In certain embodiments, the central channel can be formed by mechanical treatment such as cutting the scaffold to form the channel using a blade or laser. In other embodiments, the central channel is formed by controlling the cooling of the solution comprising fibre-forming molecules such that as the scaffold is formed, the cooling step is terminated prior to the scaffold being formed completely resulting in a central channel. Alternatively, the central channel can be formed upon cooling the fibre-forming solution using a cylindrical tube as the receptacle having an inner tube or cylinder which will define the geometry of the central channel.

The central channel can be of any suitable dimension. In certain embodiments, the central channel has a diameter greater than 0.1 mm, 0.4 mm, 0.8 mm, 1 cm or 2 cm. In certain embodiments, the central channel has a diameter of from 0.1 mm to 2 cm. In certain embodiments, the central channel has a diameter of from 0.1 mm to 1 cm. In certain embodiments, the central channel has a diameter of from 0.1 to 4 mm. In certain embodiments, the central channel has a diameter of from 0.2 to 4 mm. In certain embodiments, the central channel has a diameter of from 0.1 to 2 mm. In certain embodiments, the central channel has a diameter of from 0.4 to 2 mm. In certain embodiments, the central channel has a diameter of from 0.4 to 1 mm. In certain embodiments, the central channel has a diameter of from 0.4 to 0.8 mm.

### Cell Culture, Cell Growth and Tissue Repair

The scaffolds of the present invention can be suitable to promote cell growth, cell culture and tissue formation in the bulk 3D scaffolds. Accordingly, the cells associated with the scaffolds of the present invention have any desirable cell viability and will be determined based on the desired application. As will be understood by a person skilled in the art, the cells can be cultured on the scaffolds of the present invention using any suitable technique known in the art. Typically, the cells can be cultured on the scaffolds after formation of the scaffold.

It is to be understood that any suitable cells can be used for cell culture on the scaffolds of the present invention. The type of cell used will be determined based on the application of the scaffold. In certain embodiments, the present invention can provide a method of promoting cell growth comprising capturing and culturing cells within a scaffold of the present invention. In certain embodiments, the cell is selected from a neuronal cell, skin cell, fibroblast, vascular cell, endothelial cell, bone cell, muscle cell, cardiac cell, corneal cell, eardrum cell, cancer cell and combinations thereof. In certain embodiments, the cell is selected from a neuronal cell, fibroblast, endothelial cell, stem cell, progenitor cell and combinations thereof.

In some embodiments, the method of promoting cell growth comprises promoting nerve repair or regeneration wherein the cell is a neuronal cell. In some embodiments, the method of promoting cell growth comprises promoting blood vessel repair or formation wherein the cell is an endothelial cell.

In some embodiments, the present invention can provide use of a scaffold of the present invention in the preparation of a biomedical implant for promoting cell growth comprising capturing and culturing cells. In some embodiments, the use comprises promoting nerve repair or regeneration wherein the cell is a neuronal cell. In some embodiments, the use comprises promoting blood vessel repair or formation wherein the cell is an endothelial cell.

As will be apparent to a person skilled in the relevant art, the scaffolds can be used in any suitable application for cell culture, tissue regeneration or tissue repair. In some embodiments, the scaffold can be used as a biomedical implant. In some embodiments, the scaffolds can be used as artificial blood vessels. In certain embodiments, the scaffolds can be used to heal wounds, repair bone damage, treat damaged tissue, drug delivery or in vitro cell culture. The scaffold can be used as a substrate for in vitro cell culture by providing a coating or layer of the scaffold on cell culture dishes, plates and flasks. Advantageously, in embodiments where the fibres are radially aligned, the scaffolds can be used for tissue or wound repair as radial fibres can promote wound closure.

In one embodiment, the present invention provides a method of treating a mammal suffering from a tissue injury and in need of tissue restoration and/or regeneration, comprising applying to the injury site a scaffold of the present invention.

In one embodiment, the present invention provides use of a scaffold of the present invention in the preparation of a biomedical implant for the treatment of a tissue injury and tissue restoration and/or regeneration.

In one embodiment, the present invention provides use of a scaffold for treating a mammal suffering from a tissue injury and in need of tissue restoration and/or regeneration, comprising applying to the injury site the scaffold of the present invention.

When the scaffold or biomedical implant is used for tissue engineering or tissue restoration and/or regeneration applications, the method can be carried out, for example, by implanting the scaffold (i.e. porous biocompatible scaffold that fails to cause an acute reaction when implanted into a patient) or biomedical implant into a mammal and then removing the scaffold or biomedical implant from the mammal (such as a human). The scaffold or biomedical implant is implanted in direct contact with (i.e. physically touching over at least a portion of its external surface), or adjacent to (i.e. physically separated from) mature or immature target tissue, for a period of time that is sufficient to allow cells of the target tissue to associate with the scaffold or biomedical implant. In some embodiments, the scaffold or biomedical implant can be pre-seeded with cells of the target tissue. The tissue graft includes the removed scaffold and the associated cells of the target tissue.

“Target tissue” is tissue of any type that a graft is generated to replace. For example, where a patient has torn or otherwise damaged a ligament, and that ligament is targeted for replacement with a graft created by the methods described herein, the target tissue is ligament. When the patient has damaged cartilage, the target tissue is cartilage; when the patient has a damaged tendon, the target tissue is tendon; and so forth. The target tissue is “mature” when it includes cells and other components that are naturally found in fully differentiated tissue (e.g. a recognizable ligament in an adult mammal is a mature target tissue). The target tissue is “immature” when it includes cells that have not yet differentiated into, but which will differentiate into, mature cells (e.g., immature target tissue can contain mesenchymal stem cells, bone marrow stromal cells, and precursor or progenitor cells). Target tissue is also “immature” when it contains cells that induce immature cells to differentiate into cells of a mature target tissue or when it contains cells that sustain mature cells (these events can occur, for example, when cells secrete growth factors or cytokines that bring about cellular differentiation or sustain mature cells). Thus, the scaffold or biomedical implant of the present invention can be carried out by implanting a scaffold or biomedical implant comprising the scaffold of the present invention in direct contact with, or adjacent to, target tissue or tissue that includes cells that can produce target tissue (by, for example, the processes described herein—differentiation or through the action of growth factors or cytokines).

In some embodiments, the mammal that has the tissue defect and the mammal from which the tissue graft is obtained can be the same mammal or the same type of mammal (e.g. one human patient can have a tissue defect that is treated with a graft generated in another human). Alternatively, the mammal that has the tissue defect and the mammal from which the tissue graft is obtained can be different types of mammals (e.g., a human patient can have a tissue defect that is treated with a graft generated in another primate, a cow, a horse, a sheep, a pig, or a goat).

Once obtained, the scaffold or biomedical implant can be implanted in a mammal at the site of a tissue defect by any surgical technique. For example, the scaffold or biomedical implant can be sutured, pinned, tacked, or stapled into a mammal at the site of a tissue defect. In one embodiment, the scaffold or biomedical implant is implanted by attaching a first portion of the scaffold or biomedical implant to a first support structure at the site of the tissue defect and attaching a second portion of the scaffold or biomedical implant to a second support structure at the site of the tissue defect, such that the scaffold or biomedical implant connects the first support structure to the second support structure.

In the event the first support structure is the tibia, the second support structure can be the femur. In the event the first support structure is a first articular surface of a joint (e.g. a shoulder, wrist, elbow, hip, knee or ankle joint), the second support structure can be a second articular surface of the same joint (i.e., the shoulder, wrist, elbow, hip, knee, or ankle joint, respectively).

As used herein, the term “adjacent to” means that the scaffold or biomedical implant is separated from the tissue of the target type, or tissue comprising cells that can produce tissue of the target type or both, if both are present, by a distance of up to 10 mm and preferably less than 5 mm.

The viabilities of cells associated with the scaffolds or biomedical implants can be measured using any suitable technique known in the art. The cell viabilities can be measured using colorimetric assays, for example, MTT (3-(4,5-dimethylthiazol-2-yl)-2,5-diphenyltetrazolium bromide) assay, XTT (2,3-bis-(2-methoxy-4-nitro-5-sulfophenyl)-2H-tetrazolium-5-carboxanilide) assay, MTS (3-(4,5-dimethylthiazol-2-yl)-5-(3-carboxymethoxyphenyl)-2-(4-sulfophenyl)-2H-tetrazolium) assay, WST (Water-soluble Tetrazolium salts) assays or the like. Alternatively, the viabilities of the cells may be assessed using microscopy techniques with cell staining to differentiate between live and dead cells.

### Composite Materials

The present invention also relates to modified materials such as textile fabrics, bandages or other existing products with different types and compositions of fibres as described herein to produce a composite material such as a biomimetic composite material. In one embodiment, the present invention provides a composite material comprising a matrix of substantially aligned fibres and at least a base material.

In some embodiments, the composite material is porous. In some embodiments, the composite material is non-porous. It is to be understood that the composite materials are suitable to promote cell growth and/or tissue formation.

The composite materials of the present invention can be used for disease treatment, wound healing, tissue regeneration, drug delivery and the like. For composite materials comprising textile fabrics as the base material, properties including feeling, comfort, air-permeability, mechanical properties, antimicrobial (such as antiviral, antibacterial and antialgal) properties, hydrophobicity and hydrophilicity can be tailored. In some embodiments, the composite materials can be used as bandages or dressings for wound healing, tissue regeneration and treatment of diseases such as diabetes.

The composite material of the present invention can comprise any suitable amount of fibres. The functional aspects of the composite material including cell adhesion, proliferation, growth, differentiation, antimicrobial function, and tissue regeneration can be tailored depending on the amount of aligned fibres and the fibre-forming liquid used.

It will be appreciated by one of ordinary skill in the art that the composite materials of the present invention can comprise additives such as drugs or growth factors that may be beneficial for cell adhesion, proliferation, growth, differentiation, tissue regeneration or antimicrobial properties. In some embodiments, additives can be added into the solution of fibre-forming molecules to provide aligned fibres comprising additives loaded, adsorbed or absorbed in the composite material.

Typically, the base material is immersed in a solution of fibre-forming molecules which is then cooled using the present invention to provide the composite material having aligned fibres.

### Base Material

The base material can be any suitable material which is suitable as a template to incorporate the aligned fibres of the present invention. Examples of base materials include bandages, dressings and textile fabrics. In some embodiments, the base material can be a scaffold prepared by the method of the present invention. The base material can be of any suitable material which is porous or non-porous which can incorporate the aligned fibres in the composite material of the present invention. In certain embodiments, the base material can be porous or non-porous. In embodiments where the base material is non-porous, the aligned fibres can be formed on a surface of the base material. In embodiments where the base material is porous, the aligned fibres can be formed within in the pores and/or on the surface of the base material. When aligned fibres form on the surface of the base material, the aligned fibres can form a scaffold if there are sufficient fibre-forming molecules.

The present invention can provide aligned fibres on or in the base material more uniformly and firmly compared to techniques known to an ordinary person skilled in the art including deposition, dispersion and coating technologies. Advantageously, the present invention is facile, efficient and cost-effective for modifying various base materials at a large scale to provide the resulting composite materials.

In some embodiments, the base material is selected from the group consisting of a natural polymer, a synthetic polymer and combinations thereof.

Natural polymers may include polysaccharides, polypeptides, glycoproteins, and derivatives thereof and copolymers thereof. Polysaccharides may include agar, alginates, chitosan, hyaluronan, cellulosic polymers (e.g., cellulose and derivatives thereof as well as cellulose production by-products such as lignin) and starch polymers. Polypeptides may include various proteins, such as silk fibroin, silk sericin, lysozyme, collagen, keratin, casein, gelatin and derivatives thereof. Derivatives of natural polymers, such as polysaccharides and polypeptides, may include various salts, esters, ethers, and graft copolymers. Exemplary salts may be selected from sodium, zinc, iron and calcium salts.

In certain embodiments, the natural polymer is selected from the group consisting of at least one of silk fibroin, alginate, bovine serum albumin, collagen, chitosan, gelatin, sericin, hyaluronic acid, starch and derivatives thereof. In certain embodiments, the natural polymer is selected from the group consisting of silk fibroin, alginates, gelatin, silk fibroin/alginate, silk fibroin/bovine serum albumin, silk fibroin/collagen, silk fibroin/chitosan, silk fibroin/gelatin and derivatives thereof.

Synthetic polymers may include vinyl polymers such as, but not limited to, polyethylene, polypropylene, poly(vinyl chloride), polystyrene, polytetrafluoroethylene, poly(α-methylstyrene), poly(acrylic acid), poly(methacrylic acid), poly(isobutylene), poly(acrylonitrile), poly(methyl acrylate), poly(methyl methacrylate), poly(acrylamide), poly(methacrylamide), poly(1-pentene), poly(1,3-butadiene), poly(vinyl acetate), poly(2-vinyl pyridine), poly(vinyl alcohol), poly(vinyl pyrrolidone), poly(styrene), poly(styrene sulfonate) poly(vinylidene hexafluoropropylene), 1,4-polyisoprene, and 3,4-polychloroprene. Suitable synthetic polymers may also include non-vinyl polymers such as, but not limited to, poly(ethylene oxide), polyformaldehyde, polyacetaldehyde, poly(3-propionate), poly(10-decanoate), poly(ethylene terephthalate), polycaprolactam, poly(11-undecanoamide), poly(hexamethylene sebacamide), poly(m-phenylene terephthalate), poly(tetramethylene-m-benzenesulfonamide). Copolymers of any one of the aforementioned may also be used.

Synthetic polymers employed in the process of the invention may fall within one of the following polymer classes: polyolefins, polyethers (including all epoxy resins, polyacetals, poly(orthoesters), polyetheretherketones, polyetherimides, poly(alkylene oxides) and poly(arylene oxides)), polyamides (including polyureas), polyamideimides, polyacrylates, polybenzimidazoles, polyesters (e.g. polylactic acid (PLA), polyglycolic acid (PGA), poly(lactic-co-glycolic acid) (PLGA)), poly(lactide-co-c-caprolactone) (PLCL), polycarbonates, polyurethanes, polyimides, polyamines, polyhydrazides, phenolic resins, polysilanes, polysiloxanes, polycarbodiimides, polyimines (e.g. polyethyleneimine), azo polymers, polysulfides, polysulfones, polyether sulfones, oligomeric silsesquioxane polymers, polydimethylsiloxane polymers and copolymers thereof.

In some embodiments, functionalised synthetic polymers may be used. In such embodiments, the synthetic polymers may be modified with one or more functional groups. Examples of functional groups include Arg-Gly-Asp (RGD) peptides, boronic acid, alkyne, amino, carboxyl or azido functional groups. Such functional groups will generally be covalently bound to the polymer. The functional groups may allow the polymer to undergo further reaction, or to impart additional properties to the fibres.

In some embodiments, the base material includes a water-soluble or water-dispersible polymer, or a derivative thereof. In some embodiments, the base material comprises a water-soluble or water-dispersible polymer, or a derivative thereof. Exemplary water-soluble or water-dispersible polymers include polypeptides, alginates, chitosan, starch, collagen, polyurethanes, polyacrylic acid, polyacrylates, polyacrylamides (including poly(N-alkyl acrylamides) such as poly(N-isopropyl acrylamide), poly(vinyl alcohol), polyallylamine, polyethyleneimine, poly(vinyl pyrrolidone), poly(lactic acid), poly(ethylene-co-acrylic acid), polyesters (e.g. polylactic acid (PLA), polyglycolic acid (PGA), poly(lactic-co-glycolic acid) (PLGA)), poly(lactide-co-c-caprolactone) (PLCL), polycarbonates, polyurethanes, polypropylene) and copolymers thereof and combinations thereof. Derivatives of water-soluble or water-dispersible polymers may include various salts thereof.

In some embodiments, the base material includes organic solvent soluble polymers selected from the group consisting of poly(styrene) and polyesters such as poly(lactic acid), poly(glycolic acid), poly(caprolactone) and copolymers thereof, such as poly(lactic-co-glycolic acid).

In some embodiments, the base material includes a hybrid polymer. Hybrid polymers may be inorganic/organic hybrid polymers. Exemplary hybrid polymers include polysiloxanes, such as poly(dimethylsiloxane) (PDMS).

In some embodiments the base material includes at least one polymer selected from the group consisting of polypeptides, alginates, gelatin, chitosan, starch, collagen, silk fibroin, polyurethanes, polyacrylic acid, polyacrylates, polypropylene, polyacrylamides, polyesters, polyolefins, boronic acid functionalised polymers, polyvinylalcohol, polyallylamine, polyethyleneimine, poly(vinyl pyrrolidone), poly(lactic acid), polyether sulfone and inorganic polymers.

In some embodiments, the base material includes a mixture of two or more polymers, such as a mixture of a thermoresponsive synthetic polymer (e.g. poly(N-isopropyl acrylamide)) and a natural polymer (e.g. a polypeptide).

Examples of materials and methods for use with the method of the present invention will now be provided. In providing these examples, it is to be understood that the specific nature of the following description is not to limit the generality of the above description.

### EXAMPLES

The present invention will now be described with reference to the following examples.

### Silk Fibroin (SF) Solution Generation

Silk cocoons were boiled 4 times (20 min/time) in an aqueous 0.5% (w/v) Na₂CO₃ solution to remove sericin protein. The degummed silk fibres were rinsed with ultrapure water thoroughly to remove the residual of sercin. Following drying, they were dissolved in a mixture of CaCl₂, H₂O and CH₃CH₂OH (in a molar ratio of 1:8:2) at 65° C. to get a clear solution. Subsequently, the resulting solution was dialysed against ultrapure water (18.2 mΩ·cm) using cellulose dialysis tubes (molecular weight cut-off: 14 kDa; Sigma Aldrich, Australia) at ambient temperature for 4 days. The impurities were removed by filtering and centrifuging at 5000 rpm for 20 min. Finally, regenerated SF sponge was obtained by lyophilizing the centrifuged solution using a freeze dryer (FreeZone 2.5 Liter Benchtop Freeze Dryer; Labconco, Kansas City, Mo., USA). SF solution (2%) was obtained by dissolving 2 g of regenerated SF sponge in 100 mL ultrapure water for further use.

### 3D SF Scaffold Preparation

(a) Scaffolds with Aligned Nanofibres (AFb):

SF solution in a glass tube was directly immersed into liquid nitrogen. Target scaffolds were produced by freeze-drying the frozen samples using a freeze dryer. The fabrication scheme is shown in FIG. 2.

(b) Water-Resistant Aligned Nanofibrous Scaffolds (A F):

To make the scaffolds insoluble in water, the resulting scaffolds (AFb) above were post-treated by immersing in ethanol at ambient temperature for 12 h. Following removal of ethanol and thoroughly rinsing with the ultrapure water, AF scaffolds were obtained and re-immersed in the ultrapure water for use or further treatment.

(c) Scaffolds with Co-Aligned Nanofibres and Macrochannels (A(F&C)):

The AF scaffolds in ultrapure water above were frozen at −20° C. for 72 h. Following freeze-drying, A(F&C) scaffolds were obtained.

(d) Wb and W&Fb Scaffolds (Wb from Freezing at −20° C. and W&Fb from Freezing at −80 ° C.):

For comparison, scaffolds were also formed in freezers at −20° C. and −80° C., respectively, rather than by instant freezing with liquid nitrogen. For Wb scaffolds from −20° C., SF solution in the glass tube was frozen at −20° C. for 53 h. For W&Fb scaffolds from −80° C., SF solution in the glass tube was frozen at −80° C. for 53 h. Following removal of ice crystal by freeze-drying, Wb and W&Fb scaffolds are respectively obtained.

(e) W and W&F Scaffolds:

Wb and W&Fb scaffolds above were further processed with the same procedures for obtaining A(F&C) scaffolds, i.e., the scaffolds were post-treated by immersing in ethanol at ambient temperature for 12 h. After removing ethanol and thoroughly rinsing with ultrapure water, the scaffolds in the ultrapure water were frozen at −20° C. for 72 h. Following freeze-drying, W and W&F scaffolds were obtained, respectively.

### 3D SF/Gelatin Composite A(F&C) Scaffolds

SF/gelatin (Sigma-Aldrich, Australia) solution (2%) was obtained by dissolving 2 g of regenerated SF/gelatin mixture (in a weight ratio of 95:5) in 100 mL ultrapure water for further use. Then the SF/gelatin composite A(F&C) scaffolds were fabricated by the same protocol for producing SF A(F&C) scaffolds above.

### 3D Sodium Alginate A(F&C) Scaffolds:

Sodium alginate (Sigma-Aldrich, Australia) solution (0.3% w/v) was fabricated by dissolving 0.3 g of sodium alginate in 100 mL ultrapure water at 50° C. under stirring. Apart from the post-treatment of AFb scaffolds to form AF scaffolds using an aqueous CaCl₂ solution instead of ethanol, the sodium alginate A(F&C) scaffolds were prepared by the same protocol for fabricating SF A(F&C) scaffolds.

### Composite Material

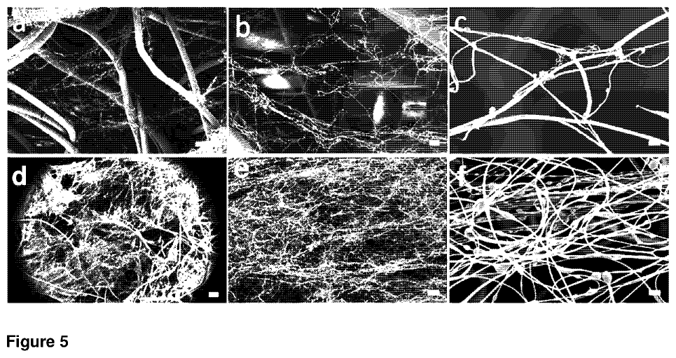

A solution of fibre-forming molecules and a base material (for example polypropylene porous microfibrous material) in a container; or a base material with an absorbed solution of fibre-forming molecules (such as silk fibroin solution, alginate solution, gelatin solution, or combination thereof) were directly immersed into liquid nitrogen or slowly lowered into liquid nitrogen to induce a temperature difference. The composite material was produced by freeze-drying the frozen samples using a freeze dryer. Optionally, to make the scaffolds insoluble in water, the resulting composite scaffolds can be post-treated by immersing in a suitable cross-linker (such as an ethanol solution) or in a vapour environment of cross-linker (such as 75% ethanol vapour). The resulting composite material was obtained by drying at room temperature or thoroughly rinsing with ultrapure water and then freeze-drying. Representative micrographs are shown in FIGS. 4 and 5.

### Characterisation

The morphology of materials was observed using a scanning electron microscopy (SEM) (Zeiss Supra 55VP), and fibre diameter was determined from representative SEM images by an image processing software (Image-J 1.34). Fourier transform infrared spectroscopy (FTIR) spectra were recorded in a wavenumber range of 600-4000 cm⁻¹ using a Bruker VERTEX 70 instrument in an attenuated total reflectance (ATR) mode (4 cm⁻¹ resolution, 64 scans). Compressive mechanical properties of silk scaffolds were obtained using an Instron 5967 Computerized Universal Testing Machine (Instron Corp, USA) with a 100 N loading cell. Cylindrical scaffolds with a diameter of 10 mm and height of 4 mm were measured at a crossing-head speed of 5 mm/min (six samples were measured for each group). Compressive stress and strain were graphed, and the compressive modulus was calculated as the slope of the initial linear section of the stress-strain curve. The architecture of silk scaffolds was imaged using Micro X-ray Computed Tomography (micro-CT) by an Xradia© micro XCT200 (Carl Zeiss X-ray Microscopy, Inc., USA). An X-ray tube with a voltage of 40 kV and a peak power of 10 W was used. 361 equiangular projections (exposure time: 8 seconds/projection) over 180 degrees were taken for one complete tomographic reconstruction. Phase retrieval tomography with 3D reconstruction algorithm was introduced to obtain clear projections and a final 3D visualization. The size of reconstructed 3D images was 512×512×512 voxels with a 4.3 μm voxel size along each side.

### Scaffold Cell Capturing, Growth and In Vitro Vascularisation of Human Umbilical Vein Endothelial Cells in 3D SF Scaffolds.

Human Umbilical Vein Endothelial Cell (HUVEC; Life Technologies, Australia) Culture and Scaffold Seeding: HUVECs were cultured in Medium 200 with Low Serum Growth Supplement (LSGS; Life Technologies, Australia). Scaffolds (diameter around 10 mm and thickness around 3 mm) were placed in 24-well plates (Greiner Bio-One) after sterilization in an environment of 75% ethanol vapour. HUVECs suspended in cell medium were evenly seeded onto scaffolds at a corresponding density (1×10⁵/well, 1.5×10⁵/well and 2×10⁵/well for in vitro cell adhesion, proliferation and vascularization study, respectively). Cell-seeded scaffolds were maintained in vitro under standard culture conditions (37° C., 5% CO₂) with medium change every 2-3 days.

(a) Cell Capture and Growth in Scaffolds:

At fixed time points (2, 4 and 8 hours for cell capture assay; 2, 4 and 6 days for cell proliferation assay) after seeding, the viability of cells on scaffolds was analysed using MTS assay (Promega, USA) following the manufacturer's instructions with absorbance measured at 490 nm on a microplate reader (SH-1000Lab, Corona Electric Co., Ltd, Japan).

Cell morphology on scaffolds was observed using confocal fluorescence microscopy (Leica TCS SP5 Confocal Microscope, Leica Microsystems, Wetzlar) after 3 days of culture. Cell-scaffold composites were rinsed with PBS, and fixed in 4% paraformaldehyde (Sigma-Aldrich, Australia) for 30 min at ambient temperature. After rinsing with PBS, the composites were permeabilized with 0.1% Triton X-100 (Sigma-Aldrich, Australia) for 10 min, followed by rinsing with PBS. The composites were then incubated in Image-iT® FX Signal Enhancer Ready Probes™ reagent (Life Technologies, Australia) for 30 min and rinsed with PBS. Subsequently, the composites were incubated with Alexa Fluor® 568 Phalloidin (1:100; Life Technologies, Australia) for 1 hour. After rinsing in PBS, the composites were incubated in DAPI (Life Technologies, Australia) in the dark for 10 min. As-treated samples were assessed using the confocal fluorescence microscope.

(b) In Vitro Vascularisation in Scaffolds:

After 21 d of culture, cell-scaffold composites were rinsed with PBS, and fixed in 4% paraformaldehyde (Sigma-Aldrich, Australia) for 30 min at ambient temperature. Following rinsing with PBS, the composites were permeabilized with 0.1% Triton X-100 (Sigma-Aldrich, Australia) for 10 min, followed by rinsing with PBS. The composites were then incubated in Image-iT® FX Signal Enhancer Ready Probes™ reagent (Life Technologies, Australia) for 30 min. After rinsing with PBS, the composites were incubated for 10 min with 10% Normal Goat Serum blocking solution (Life Technologies, Australia) to block non-specific binding and then rinsed with PBS. Subsequently, the composites were incubated with CD31 Monoclonal Antibody (1:50; Life Technologies, Australia) overnight at 4° C. Following rinsing with PBS, the composites were incubated with Goat-anti-Mouse IgG (H+L) Secondary Antibody, Alexa Fluor® 488 conjugate (1:200; Life Technologies, Australia) for 1 hour. The scaffolds were rinsed again with PBS and incubated in DAPI (Life Technologies, Australia) in dark for 10 min. As-treated samples were assessed using the confocal fluorescence microscope.

### Scaffold Cell Capturing and Neurite Outgrowth of Rat Embryonic Dorsal Root Ganglion Neurons in 3D SF Scaffolds.

(a) Rat Embryonic Dorsal Root Ganglion Neuron (DRG; Lonza, USA) Culture and Scaffold Seeding:

DRGs were cultured in Primary Neuron Basal Medium (PNBM; Lonza, USA) supplemented with PNGM™ SingleQuots™ (Lonza, USA) and 150 ng/ml of Nerve Growth Factor (NGF; Sigma-Aldrich, Australia).

Scaffolds (diameter around 10 mm and thickness around 3 mm) were placed in 24-well plates (Greiner Bio-One) after sterilization with 75% ethanol vapour. DRGs suspended in cell medium were evenly seeded onto scaffolds at a density of 1.2×10⁵/well. DRGs-seeded scaffolds were maintained in vitro under standard culture conditions (37° C., 5% CO₂) with medium change every 3-5 days.

(b) Cell Capturing of Scaffolds:

At fixed time points (6, 12 and 24 h) after seeding, the viability of DRGs captured by scaffolds was analysed using MTS assay (Promega, USA) following the manufacturer's instructions with absorbance measuring at 490 nm on a microplate reader (SH-1000Lab, Corona Electric Co., Ltd, Japan).

(c) Immunostaining for Neurite Outgrowth of DRGs:

After 21 days of culture, the scaffolds were rinsed with PBS and fixed in 4% paraformaldehyde (Sigma-Aldrich, Australia) for 30 min at ambient temperature. Following rinsing with PBS, the composites were permeabilized with 0.1% Triton X-100 (Sigma-Aldrich, Australia) for 30 min and then rinsed with PBS again. Subsequently, the scaffolds were incubated in 10% Normal Goat Serum blocking solution (Life Technologies, Australia) for 10 min to block non-specific binding, followed by rinsing with PBS. Then the scaffolds were incubated with Anti-Neurofilament-200 antibody from rabbit (1:50; Sigma-Aldrich, Australia) overnight at 4° C. After rinsing with PBS, the scaffolds were incubated with Goat-anti-Rabbit IgG (H+L) Secondary Antibody, Alexa Fluor® 488 conjugate (1:200; Life Technologies, Australia) for 1 hour. Finally, the treated samples were analysed using the confocal fluorescence microscope.

(d) Statistical Method:

All experiments were carried out in triplicate and data were expressed as mean±standard deviation (SD). Statistical differences were analysed by one-way ANOVA using statistical software in the Origin 9 software package (OriginLab, USA). Difference with p<0.05 or p<0.01 was considered as statistical significance.

### Example 1

### Silk Fibroin Scaffolds

FDA-approved silk fibroin (SF) has been widely recognized and used as biomedical materials due to its excellent biocompatibility, tunable mechanical properties, biodegradability and low inflammatory response. With silk fibroin as a model, the inventors demonstrated that 3D silk fibroin (SF) scaffolds with co-aligned nanofibres and macrochannels can be conveniently created by a facile freeze-drying approach. The method is based on control of ice crystallisation. When a volume of water is frozen, the size of ice crystals and their orientation are controlled by a temperature gradient, freezing speed, and the direction of the temperature gradient of the volume. A lower temperature and faster freezing, i.e. a higher thermodynamic driving force and kinetics, promote ice nucleation, leading to a larger number of fine crystals.

On the basis of this principle, the inventors adopted a two-step freezing method to create the desired SF structures using various fibre-forming molecules such as silk fibroin, a mixture of silk fibroin/gelatin and sodium alginate. The general schematic is shown in FIG. 2. Firstly, a tube containing aqueous SF solution was immersed into liquid nitrogen quickly. The extremely low temperature (around −196° C. in liquid nitrogen) and large temperature difference along the radial direction of the tube led to formation of radially aligned fine ice crystals. Radially aligned SF nanofibres, i.e., along the ice crystal growth direction, were obtained after removing ice crystals with freeze-drying.

After fixing the structure of the protein nanofibres using ethanol, i.e., to make the nanofibres insoluble in water, the nanofibrous scaffold was put in water and frozen again but at a higher temperature of −20° C. This relatively higher temperature led to the formation of larger ice crystals which were directed by radially aligned nanofibres to grow along the direction of fibres. The formation of the crystals reduces the free space for nanofibres, which pushes and squeezes the nanofibres to around the crystals. After removing these crystals with freeze-drying, macrochannels with nanofibrous walls are created in the aligned 3D nanofibrous scaffolds. In comparison with the widely used 3D silk scaffolds, the present inventive 3D scaffolds with co-aligned nanofibres and macrochannels can capture more cells that are both adherent and non-adherent. More interestingly, the scaffolds not only significantly promote cell proliferation, but also direct Human Umbilical Vein Endothelial Cells (HUVECs) to assemble into vessel-like structures and the 3D growth of Embryonic Dorsal Root Ganglion Neurons (DRGs) and neurites.

### Example 2

### Formation of 3D Architectures with Co-Aligned Nanofibres and Macrochannels

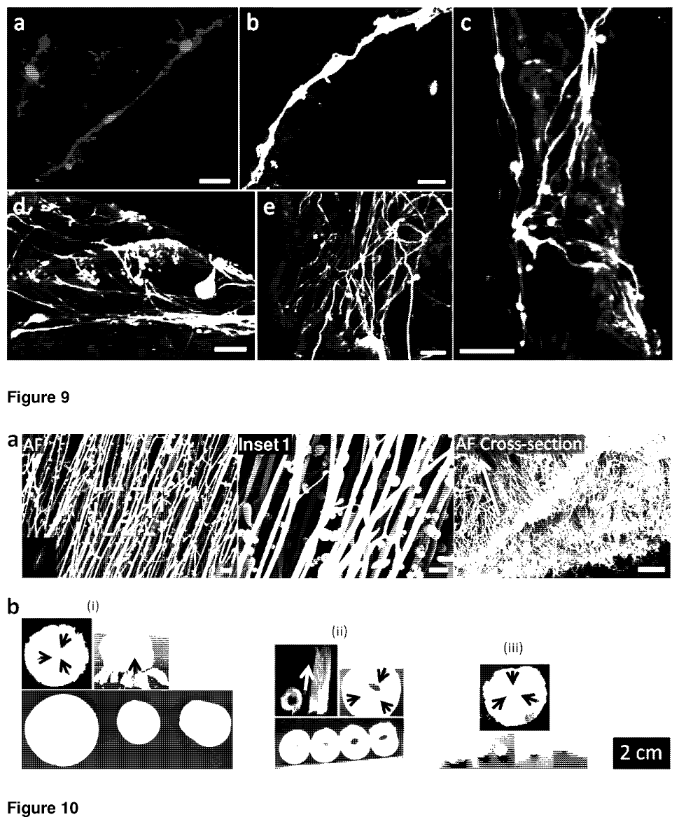

During the first freezing in liquid nitrogen, silk fibroin (SF) molecules assembled between fine ice crystals were radially oriented (FIG. 2a). After removing the ice crystals in the frozen samples by freeze-drying, 3D SF scaffolds, namely, AFb scaffolds (FIG. 2b) with radially aligned nanofibres and uniformly distributed nanoparticles were obtained (see FIG. 10a). In the following study, the radially aligned 3D nanofibrous scaffolds without channels before post-treatment in ethanol are indicated as AFb (A, F and b respectively represent ‘aligned’, ‘nanofibres’ and ‘before post-treatment in ethanol’, respectively). Clearly, as-prepared SF nanofibres presented a smooth morphology and were well aligned radially (see FIG. 10a). This method is facile and allows the fabrication of samples with varied geometries (even including tubes and particles), diameters and thicknesses (see FIG. 10b). Furthermore, the alignment direction of scaffold nanofibres can be controlled by directionally freezing SF solution in liquid nitrogen (see FIG. 10b). For example, vertically aligned nanofibres can be fabricated by slowly lowering the SF solution-containing tube into liquid nitrogen. By directly dropping SF solution into liquid nitrogen, particles with radially aligned nanofibres were obtained. Additionally, dripping or spraying the solution comprising fibre-forming molecules (silk fibroin) into liquid nitrogen produced particles or spheres with radially aligned nanofibres similar to that of FIG. 10b.

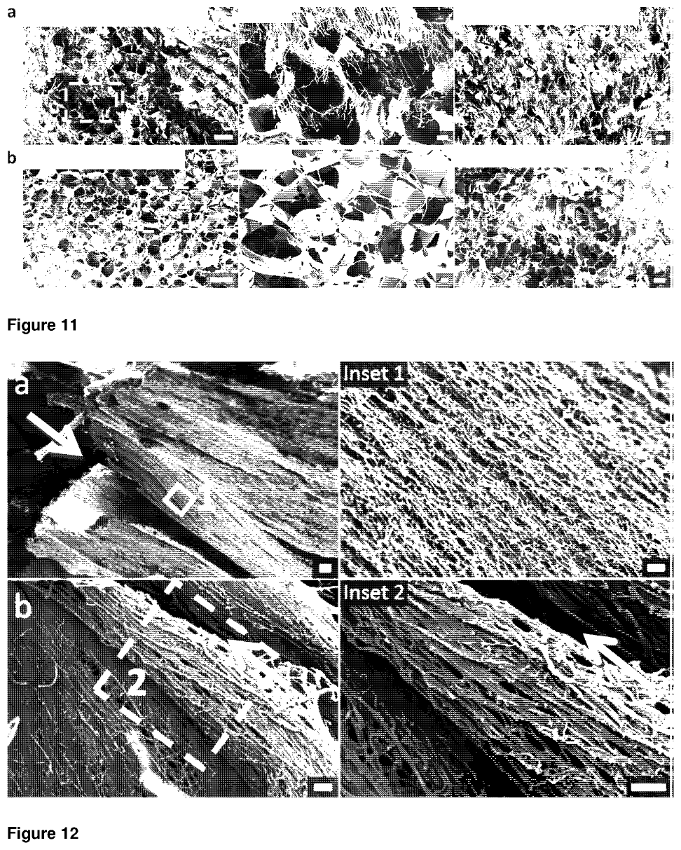

The present inventors showed that fast freezing and a high temperature difference are beneficial to the formation of nanofibres and directional structures. Instead of using liquid nitrogen for instant freezing, SF solutions contained in the same glass tubes were frozen in freezers at −80° C. and −20° C., respectively, followed by the removal of ice crystals using freeze-drying. SF scaffolds from −80° C. freezing have a hybrid structure with random short channel-like structures, pores and nanofibres, but these structures are not interconnected (see FIG. 11a) (In the following study, the hybrid 3D SF scaffolds from −80° C. freezing before post-treatment in ethanol are indicated as W&Fb where W represents the walls of the channels and pores, F represents the nanofibres, b indicates before post-treatment in ethanol. After post-treatment in ethanol, the scaffolds are indicated as W&F.).

In comparison, only random pores were seen in the SF scaffolds from −20° C. freezing and the pores are not well connected to form a network (also see FIG. 11b). Decreasing the freezing rate and temperature difference may lead to the growth of random and large ice crystals, facilitating the formation of large but not interconnecting pores, and hence the scaffold has a wall-like structure. In the following study, the porous wall-like 3D scaffolds from −20° C. freezing before post-treatment in ethanol are indicated as Wb where W represents the walls of pores and b indicates before post-treatment in ethanol. After post-treatment in ethanol, the scaffolds are indicated as W.

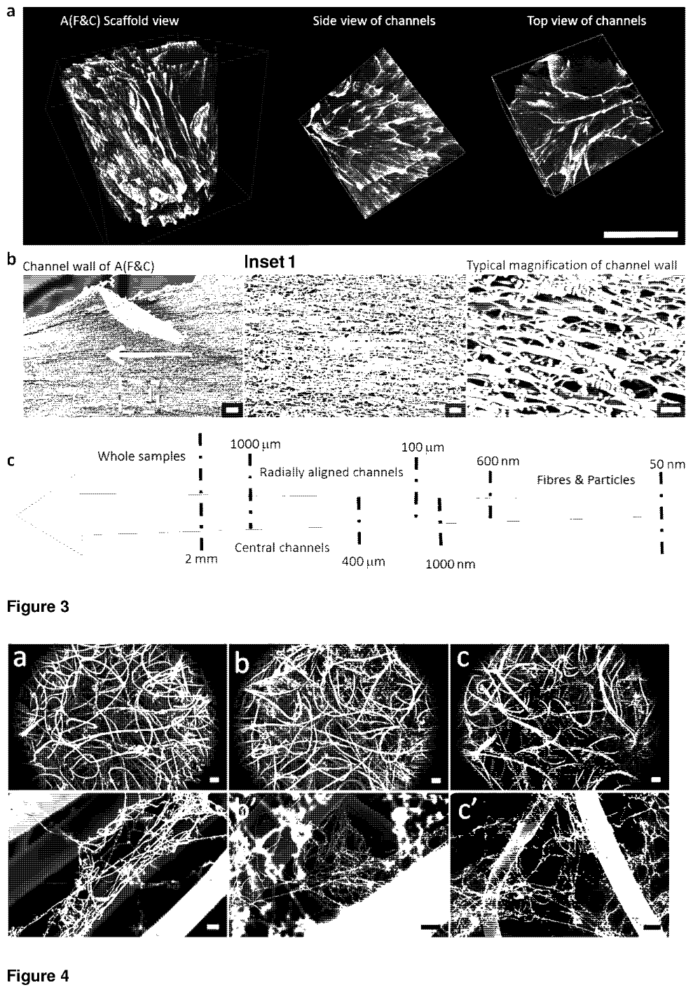

The second freezing at a lower temperature (−20° C.) created macrochannels in the fibrous scaffolds (FIG. 2c,d). From 3D micro-CT images (FIG. 3a), each radially aligned channel (diameter, 100-1000 μm) connected the surface and centre of scaffold. As shown by SEM (FIG. 3b), the channel walls are composed of SF nanoparticles and nanofibres (diameter, 50-600 nm) aligned along the direction of channels (indicated by large yellow arrows). Zooming in on a representative channel wall, many pores (diameter, 50-1000 nm) were seen which appeared to align in the orientation of nanofibres.

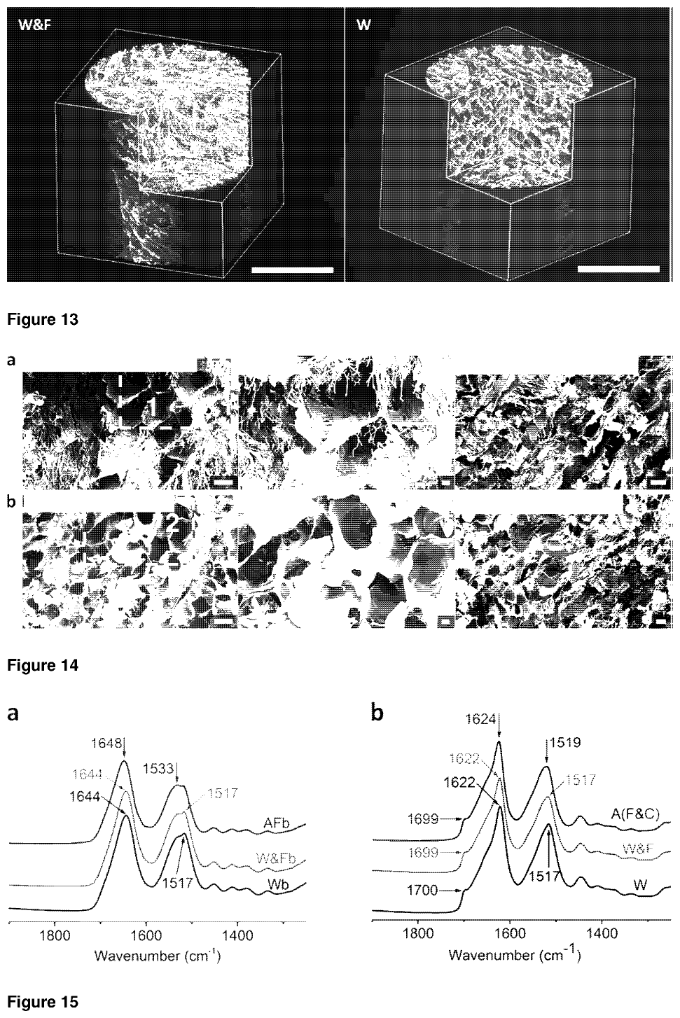

In the following study, these kinds of 3D SF scaffolds with radially aligned nanofibres and channels are indicated as A(F&C) (FIG. 2d) where A represents ‘radially aligned’, F represents the nanofibres and C represents the channels. More interestingly, a central channel (diameter, 0.4-2 mm) from the top to the bottom of scaffold were created (FIG. 2d, the digital photo of A(F&C) scaffolds). All the relevant sizes within the hierarchical 3D A(F&C) scaffolds were summarized in FIG. 3c. Interestingly, not only SF, A(F&C) scaffolds from other mixtures such as SF/gelatin as well as other biomacromolecules such as sodium alginate can also be prepared using the method of the present invention (see FIG. 12). In comparison, there was no significant change after Wb and W&Fb scaffolds being treated by the same post-treatments. The pores or short channel-like structures in both scaffolds did not appear to be interconnected (in the following study, the 3D Wb and W&Fb scaffolds after being post-treated in ethanol with the above procedure are indicated as W and W&F, respectively) (see FIGS. 13 and 14 for W&F and W scaffolds).

### Example 3

### Secondary Structure and Mechanical Characteristics of Scaffolds

Secondary structures of the scaffolds were investigated to understand the effect of preparation method on structural change of silk fibroin. It is known that the conformation change of SF can be indicated by the shift of characteristic absorption peaks (1600-1500 cm⁻¹ for amide II and 1700-1600 cm⁻¹ for amide I) in ATR-FTIR spectra. All three scaffolds before post-treatment with ethanol showed one main characteristic peak at around 1644 cm⁻¹ suggesting random coils (see FIG. 15a). The Wb and W&Fb scaffolds showed another main characteristic peak at 1517 cm⁻¹ (indicating dominant β-sheet structure), whereas the AFb scaffold showed another main characteristic peak at 1533 cm⁻¹ (indicating dominant random coil structure), suggesting the low temperature treatment with liquid nitrogen could be beneficial for the formation of random coils (see FIG. 15a). After being treated in ethanol, all three scaffolds presented main characteristic peaks at around 1700, 1622 and 1517 cm⁻¹, suggesting the treated scaffolds mainly consisted of β-sheet structure (see FIG. 15b).

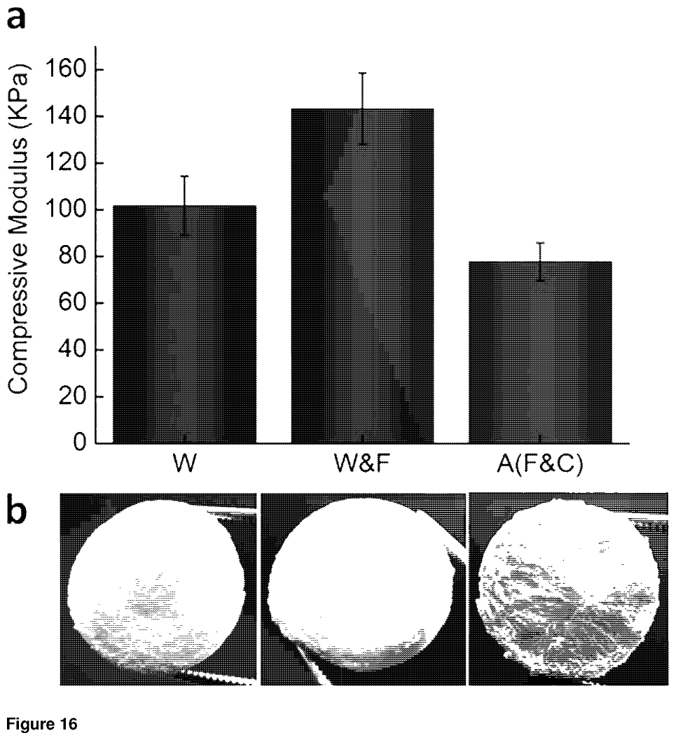

Compressive modulus of scaffolds was demonstrated in FIG. 16a. 3D A(F&C) nanofibrous scaffolds have a compressive modulus of around 80 kPa, which is lower than those of the wall-like W and W&F scaffolds (around 100 and 140 kPa, respectively). This could be due to their large channel-based structure with nanofibres. Noteworthy, after being compressed in the mechanical test, A(F&C) scaffolds still maintained a good radially aligned morphology and structure, with just some minor collapses seen on their surface, probably due to damage of some channels (see FIG. 16b).

### Example 4

### Co-Aligned Channels and Nanofibres Enhance Cell Capture, and Direct Growth, Behaviour and Function of Adherent Human Umbilical Vein Endothelial Cells (HUVECs) in 3D SF Scaffolds

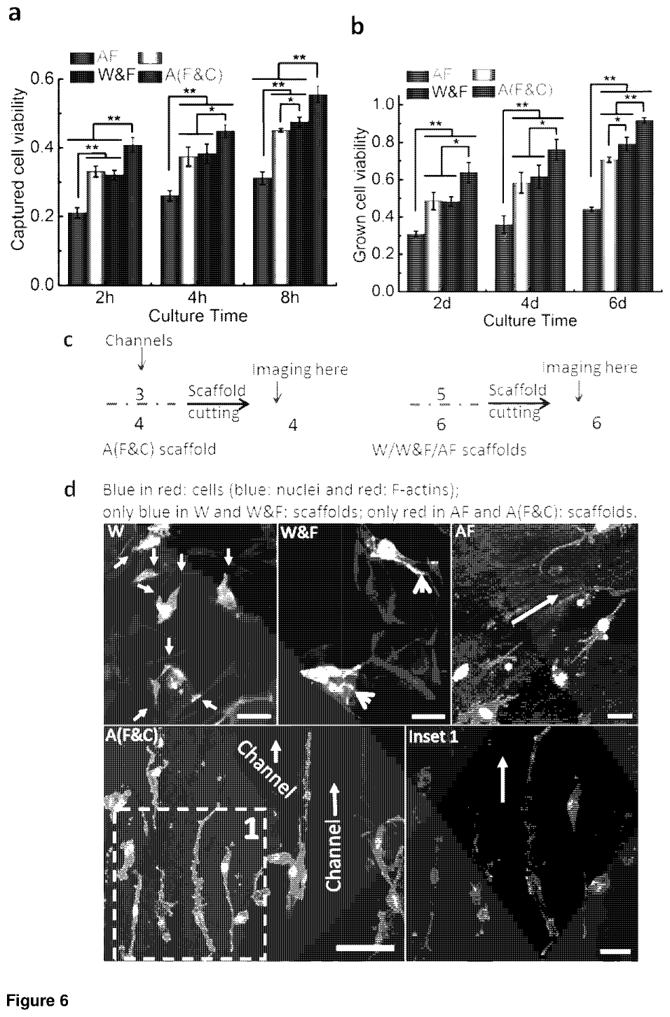

To understand the effects of aligned channels and nanofibres on cells, the ability of the scaffolds to capture cells and promote their growth was investigated using the classic adherent HUVECs. At all time points, A(F&C) scaffolds demonstrated significantly higher capacity of cell capturing and proliferation than W and W&F scaffolds, indicating that the aligned channel and nanofibrous structure of A(F&C) scaffolds are beneficial to cell adhesion and proliferation (FIG. 6a,b). Compared with W scaffolds, W&F scaffolds showed a higher cell adhesion at 8 hours and proliferation viability on day 6, which was probably due to the presence of nanofibres in W&F scaffolds.

To further identify the effect of channels, the AFb scaffolds after post treatment in ethanol (namely AF scaffolds in FIG. 6) were used as cell culture substrates. Without the second freezing step and freeze-drying, AF scaffolds had the same radially aligned nanofibous structure as AFb scaffolds shown in FIG. 2 and FIG. 10a, but they did not have channels as presented in the A(F&C) scaffolds. A(F&C) scaffolds demonstrated significantly higher cell viability than AF scaffolds at all time points, demonstrating the advantages of channels in cell capturing and proliferation. Furthermore, even W and W&F scaffolds also showed higher cell viability in comparison with AF scaffolds. This is probably due to the fact that W, W&F and A(F&C) scaffolds provide more space for cell adhesion and proliferation due to their larger pores or channels.

To gain more insight into the effects of aligned channels and nanofibres, cells grown in scaffolds for 3 days were imaged using confocal fluorescence microscopy (FIG. 6d). To date, it remains a problem that cell behaviours including cell spreading, migration, elongation and interaction are often hindered, due to the small pores and low interconnectivity of scaffolds as well as the absence of binding and guiding cues in a scaffold. This is also true to both the W and W&F scaffolds. As shown in FIG. 6d, cell spreading was significantly limited by pore walls (indicated by yellow arrows in W) or presented with blunt edges (indicated by white arrows in W&F) as if cells were cultured on surface of a flat material. Although cells were also observed in AF scaffolds (FIG. 6d), it was difficult to find them during scanning under confocal microscopy due to the small number of cells in the inner (internal) region of the scaffold. Cells in AF scaffolds were not well aligned and elongated in the direction of nanofibres, exhibiting relatively flat and polygonous morphology. This is probably due to the fact that the loosely aligned nanofibres provide cells with many surrounding signals from different directions. Cells on the walls of A(F&C) scaffolds were elongated and aligned along with nanofibres well. The presence of large 3D channels reduce spaces in the scaffolds so that nanofibres are compacted on walls of the channels, providing cells with more signals in the long-axis (longitudinal) direction of nanofibres (the direction of channels and nanofibres was indicated by white arrows, respectively). This could explain the cell growth and morphologies observed in A(F&C) scaffolds.

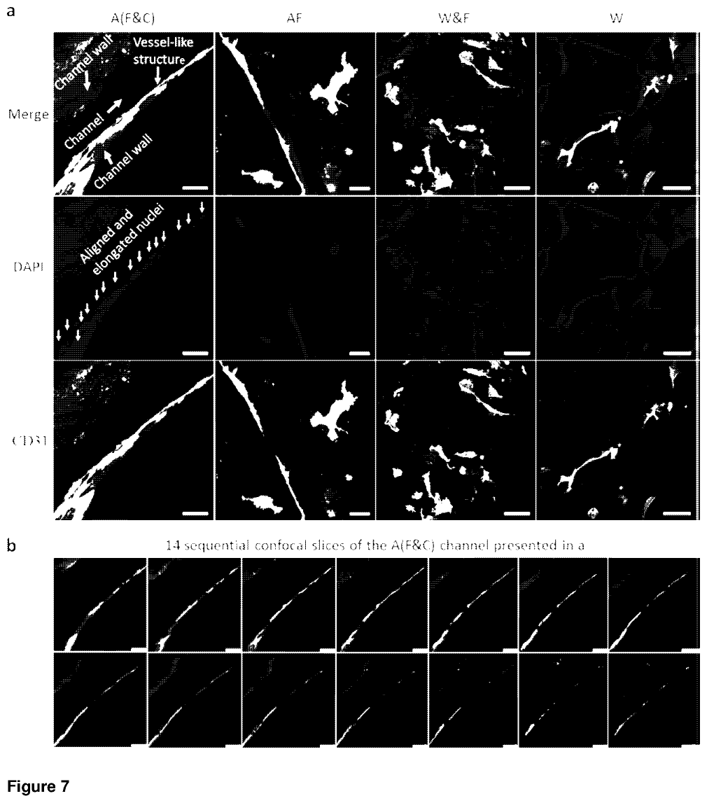

Proliferation, migration and interaction of endothelial cells are very important for the formation of tubal structures in both vasculogenesis and angiogenesis. HUVECs are a classic endothelial cell model for studying vascularization. As observed above, A(F&C) scaffolds can promote the proliferation of HUVECs. It is believed that the cell migration and elongation induced by aligned channels and nanofibres should enhance the intercellular interaction to facilitate formation of vessel-like structures. To show this, the present inventors cultured HUVECs up to 21 days to observe the vascularization behaviours of cells in the scaffolds (FIG. 7, FIG. 6c illustrates how to read the images). All cells were CD31-positive (CD31 is a glycoprotein expressed on endothelial cells), suggesting they still maintained the characteristics of HUVECs in the scaffolds after a long term of culture.

In W and W&F scaffolds, many cells still maintained the round morphology with just a few nuclei elongated (FIG. 7a). The spreading, migration and elongation of cells were limited by scaffold walls, leading to local aggregation and interaction of some cells. In AF scaffolds, although some cell nuclei were elongated, most of cells were not significantly aligned and elongated, presenting polygonous morphology (FIG. 7a). Interestingly, in A(F&C) scaffolds, all cells and cell nuclei were elongated and aligned on the wall of channels where they interacted and assembled into CD31-positive vessel-like structures (the channel, channel wall, vessel-like structures as well as aligned and elongated cell nuclei were indicated by white arrows, respectively) (FIG. 7a). Fourteen sequential confocal slices of the channel in FIG. 7a were presented in FIG. 7b. There were many vessel-like structures aligned on the wall of channel in the inner of A(F&C) scaffolds. These findings demonstrated the co-aligned channels and nanofibres enhanced spreading, migration, elongation and interaction of HUVECs to assemble the vessel-like structures.

### Example 5

### Co-Aligned Channels and Nanofibres Enhance the Capturing of Scaffolds for Non-Adherent Embryonic Dorsal Root Ganglion Neurons (DRGs) and Induce 3D Growth of Neurites in the Scaffolds

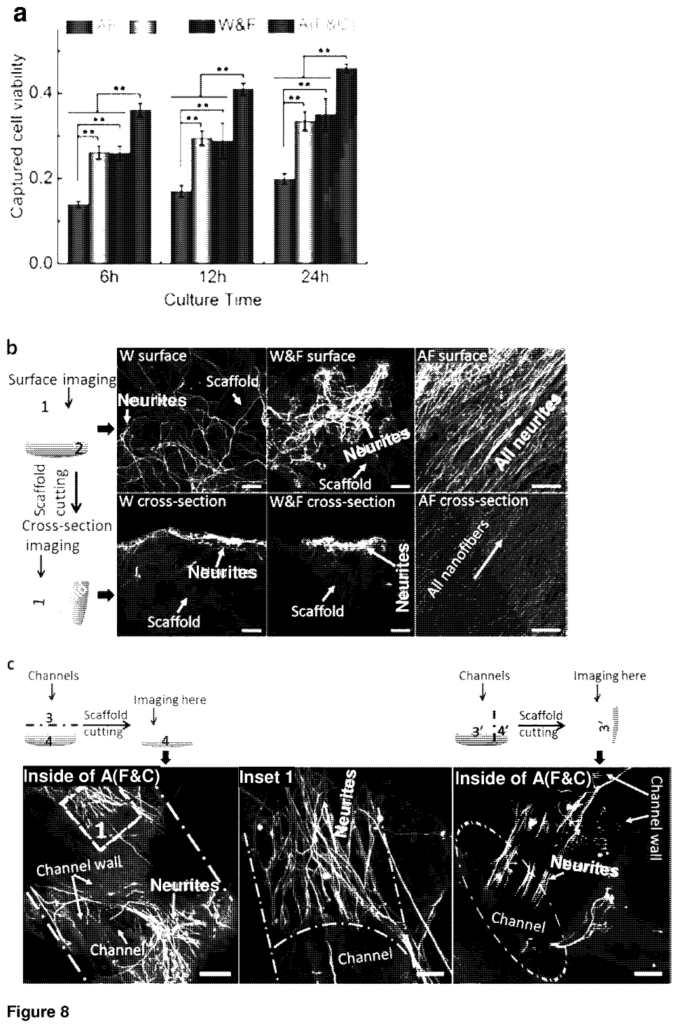

The effect of co-aligned channels and nanofibres on cells was further confirmed using non-adherent DRGs. As shown in FIG. 8a, A(F&C) scaffolds also demonstrated superior DRG capturing capacity. AF scaffolds showed the lowest DRG capturing, and no significant difference was observed between W and W&F scaffolds. These observations suggest that co-aligned channels and nanofibres of a scaffold can help capture not only adherent cells but also non-adherent cells. FIG. 8b illustrates the areas of scaffolds that were scanned and the corresponding images. Affluent neurites were aligned in the direction of nanofibres on the surface of AF scaffolds, but they were not observed in the inner portion of the scaffolds. In the macroporous W and W&F scaffolds, neurites were also aggregated on the surface only. These results indicated that in the absence of channels, it is difficult for DGRs to grow into the inner region of scaffolds during 21 days of culture and the neurite outgrowth of DRGs was suppressed.

FIG. 8c illustrates the scanned areas of A(F&C) scaffolds and the corresponding images. DRGs can be clearly seen, and a significant amount of long neurites had grown through the channel (the channels, channel walls and neurites were indicated by white arrows, respectively). Interestingly, zooming in on the channel revealed that all DRGs and neurites were mainly growing along the channels, suggesting a 3D growth mode of neurites. This was totally different from the 2D growth of DRGs and neurites along the aligned nanofibres on the surface of AF scaffolds (FIG. 8b). From the last image in FIG. 8c, neurites in bundles were observed clearly, which is very important for the formation of nerve tissues. These observations demonstrated that the aligned channels and nanofibres can not only promote the adhesion and proliferation of both adherent and non-adherent cells, but also direct them to grow, migrate and interact in the 3D space similar to the nature ECM.

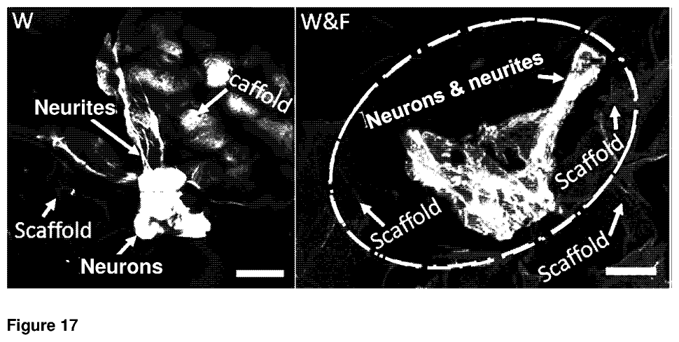

3D scaffolds with mainly wall-like porous scaffolds are currently the most investigated to date. In spite of the adjustable pore size, the low interconnectivity of pores in the scaffolds limits infiltration, migration and growth of cells and tissues as well as the transport of oxygen, nutrients and wastes. FIG. 17 provides insight into the growth of DRGs in different porous scaffolds after 21 days of culture. In W scaffolds, the neurite infiltration of aggregated DRGs happened along the pore walls only. In W&F scaffolds, the pore walls led to the aggregation of DRGs and limited the neurite outgrowth. In the A(F&C) scaffold, radially aligned channels (diameter, 100-1000 μm) towards the centre of scaffolds provided enough space for the migration and 3D growth of cells, as shown in FIG. 6d, FIG. 7a,b and FIG. 8c.

A common issue in tissue engineering is the necrosis of cells or tissues in the 3D scaffolds due to insufficient supply of oxygen and nutrients. The channels with porous walls (diameter of pores: 50-1000 nm) in the A(F&C) scaffolds are very important for the transport of oxygen, nutrients and wastes. The large central channel (diameter, 0.4-2 mm) of the scaffold should also facilitate nutrient exchange and waste disposal.

Aligned nanofibres (diameter, 50-600 nm) on channel walls played an important role in cell capturing, proliferation and directing cells to migrate and grow along the alignment direction (FIG. 6a,b,d, FIG. 7a,b and FIG. 8a). Furthermore, nanofibres and nanoparticles are good carriers for the delivery of growth factors or drugs. As shown in FIG. 7a and FIG. 8c, the channels still showed good morphology and structure after 21 days of cell culture, indicating the stability of scaffolds.

A(F&C) scaffolds were developed as a model platform for proof-of-concept that the creation of ECM-mimicking 3D structure plays an important role in insight into cell behaviours and functions in vitro. Based on this platform, the present inventors found that adherent HUVECs preferred to grow along the materials in 3D scaffolds. Therefore, they were mainly directed by the aligned nanofibres on the wall of A(F&C) scaffolds (FIG. 9a,b). In contrast, non-adherent DRGs and neurites preferred to grow along the 3D space. As shown in FIG. 9c,d,e, the neurites mainly grew along the channels. However, on the 2D surface of AF scaffolds, the neurites were highly aligned in the direction of aligned nanofibres (FIG. 8b). Considering the facile fabrication technology, the discovery in this work will pave the way for developing new types of 3D scaffolds based on aligned nanofibres and channels for use in tissue engineering. For example, creating nanofibrous tube scaffolds with radially aligned channels using biocompatible polymers could be beneficial to multi-layers of cell seeding. Likewise, constructing column scaffolds containing aligned channels in the long-axis (longitudinal) direction of scaffolds could provide a better support for nerve regeneration than hollow tubes with thin walls.

Accordingly, the present inventors have developed a facile freeze-drying strategy for creating biomimic 3D scaffolds with aligned nanofibres and macrochannels. As a model platform for cell culture and study in vitro, the 3D scaffolds showed significantly higher cell capturing and proliferation-promoting capability than widely-used wall-like 3D scaffolds and 3D aligned nanofibrous scaffolds without channels for both adherent HUVECs and non-adherent DRGs. More importantly, aligned nanofibres and channels not only direct the growth, migration, and interaction of HUVECs to assemble into blood vessel-like structures in the scaffolds in vitro, but also direct the neurite growth of DRGs in the 3D space.

Those skilled in the art will appreciate that the invention described herein is susceptible to variations and modifications other than those specifically described. It is understood that the invention includes all such variations and modifications which fall within the spirit and scope of the present invention.

1. A method for preparing a scaffold, said method comprising the steps of:
providing a solution comprising fibre-forming molecules;
subjecting the solution to a cooling medium to establish a temperature difference at an interface between the cooling medium and solution; and
cooling the solution as a result of the temperature difference to induce solvent crystallisation and alignment of fibres in the scaffold.

2. A method according to claim 1, wherein the temperature difference is sufficient to promote nucleation of solvent crystals at the interface.

3. A method according to claim 1, wherein the temperature difference is in a range of from −20° C. to −296° C. relative to the solution.

4. A method according to claim 1, wherein the cooling medium is at a temperature of from −80° C.

5. A method according to claim 1, wherein the fibres are aligned from the interface between the solution and cooling medium.

6. A method according to claim 1, wherein the temperature difference is established circumferentially to the solution to induce radially aligned fibres in the scaffold.

7. A method according to claim 1, wherein the temperature difference is established along a plane of the interface to induce linearly or longitudinally aligned fibres in the scaffold.

8. A method according to claim 6, wherein the solution is subjected by immersion in the cooling medium at a rate of 1 to 15 mm·min⁻¹.

9. A method according to claim 1, wherein the diameter of the fibre is from 20 to 5000 nm.

10. A method according to claim 1, wherein the scaffold has pores of diameter from 1 nm to 500 μm.

11. A method according to claim 1, wherein the solution further comprises an additive.

12. A method according to claim 1, further comprising subjecting the scaffold to a solution followed by an additional cooling step to induce solvent crystallisation and channels in the scaffold.

13. A scaffold prepared by the method according to claim 1.

14. A porous biomimetic scaffold comprising:
a matrix of substantially aligned fibres; optionally wherein the diameter of the fibre is from 20 to 1000 nm; and optionally further comprising channels in the scaffold for cell growth.

15.-16. (canceled)

17. A porous biomimetic scaffold according to claim 14, wherein the scaffold further comprises an additive selected from the group consisting of a drug, growth factor, polymer, surfactant, chemical, particle, porogen and combinations thereof.

18. A biomedical implant comprising a scaffold according to claim 14.

19. A method of promoting cell growth, said method comprising capturing and culturing cells with a scaffold according to claim 14.

20. A method of treating a mammal suffering from a tissue injury and in need of tissue restoration and/or regeneration, comprising applying to the injury site a scaffold according to claim 14.

21.-22. (canceled)

23. A composite material comprising:
a matrix of substantially aligned fibres; and
a base material.

24. A method according to claim 7, wherein the solution is subjected by immersion in the cooling medium at a rate of 1 to 15 mm·min⁻¹.
